(19) United States
(12) Patent Application Publication
(10) Pub. No.: US 2017/0112877 A1
(43) Pub. Date: Apr. 27, 2017

(54) NON-TOXIC AGENT FOR A BROAD-SPECTRUM, BACTERICIDAL OR BACTERIOSTATIC TREATMENT OF ANTIBIOTIC-RESISTANT BACTERIA IN ANIMALS

(71) Applicants: Alexander L. HUANG, Menlo Park, CA (US); Gin WU, San Rafael, CA (US); LIVELEAF, INC., San Carlos, CA (US)

(72) Inventors: Alexander L. Huang, Menlo Park, CA (US); Gin Wu, San Rafael, CA (US)

(21) Appl. No.: 15/317,616

(22) PCT Filed: Jun. 15, 2015
(86) PCT No.: PCT/US15/35842

§ 371 Date: Dec. 9, 2016

(51) Int. Cl. A61K 33/40; A61K 31/192; A61K 31/7032; A61K 31/353; A61K 31/7024

(52) U.S. Cl. CPC A61K 33/40; A61K 31/353; A61K 31/7024; A61K 31/7032; A61K 31/192

(57) ABSTRACT

Methods and compositions are provided for a broad-spectrum, bactericidal or bacteriostatic treatment of antibiotic-resistant bacteria in animals with a non-toxic agent. The teachings include bactericidal or bacteriostatic treatment of spore-forming, anaerobic antibiotic-resistant bacteria. And, the compositions and methods provided herein can at least inhibit the onset of, inhibit the growth of, inhibit the germination of, or kill the antibiotic-resistant bacteria. Such antibiotic-resistant bacteria include, but are not limited to, Clostridium difficile, Enterococcus faecalis, Staphylococcus aureus, and Klebsiella pneumoniae.

### CROSS-REFERENCE TO RELATED APPLICATIONS

This application is a national stage entry from International Application No. PCT/US2015/035842, filed Jun. 15, 2015, and claims the benefit of prior U.S. application Ser. No. 14/304,812, filed Jun. 13, 2014, each of which is hereby incorporated herein by reference in its entirety.

### BACKGROUND

Field of the Invention

The teachings provided herein relate to methods and compositions for a broad-spectrum, bactericidal or bacteriostatic treatment of an antibiotic-resistant bacterial infection in animals with a non-toxic agent.

Description of Related Art

Antibiotic resistance is a serious and growing problem in contemporary medicine. In fact, it is considered one of the pre-eminent public health concerns of the 21st century. Resistance to first-line antibiotics necessitates the use of second-line agents that are broader in spectrum, higher risk, more expensive and, often, locally unavailable. Any use of antibiotics can increase selective pressure in a population of bacteria to allow the resistant bacteria to thrive and the susceptible bacteria to die off. However, despite a push for new antibiotic therapies, there has been a continued decline in the number of newly approved drugs and a greater need for alternative treatments. Some carbapenem-resistant Enterobacteriaceae (CRE) bacteria, for example, have become resistant to most available antibiotics. Infections with these germs are very difficult to treat, and can be deadly. One report cites they can contribute to death in up to 50% of patients who become infected.

Examples of antibiotic-resistant bacteria include endospores such as, for example, Bacillus and Clostridium. Endospores are particularly problematic, as they can maintain dormancy and survive without nutrients. They are resistant to ultraviolet radiation, desiccation, high temperature, extreme freezing and chemical disinfectants. Common anti-bacterial agents that work by destroying vegetative cell walls do not affect endospores. Endospores are commonly found in soil and water, where they may survive for long periods of time. Astrophysicist Steinn Sigurdsson said “There are viable bacterial spores that have been found that are 40 million years old on Earth—and we know they're very hardened to radiation.”

Patients having the most risk to infections by such antibiotic-resistant bacteria are those with prior exposure to antibiotics, subjected to gastrointestinal surgery, and extended stays in healthcare settings. Those at the greatest risk are older adults, and particularly those who are immunocompromised. The bacteria of particular importance are the spore-forming, anaerobic antibiotic-resistant bacteria, such as Clostridium difficile (“C. diff”). C. diff was identified as part of normal human gastrointestinal flora in 1935, and was associated only with occasional infection episodes until the 1980s and 1990s when cases of antibiotic-associated diarrhea were proven to be caused by C. diff infection. C. diff-associated infection is the most serious form of antibiotic-associated diarrhea, the primary pathogen responsible for antibiotic-associated colitis and for 15%-25% of cases of nosocomial antibiotic-associated diarrhea. C. diff affects over 3 million patients per year, is linked to 14,000 deaths each year in the USA in those with C. diff-related diarrhea, and is associated with healthcare costs approaching $1 billion annually. Interestingly, several studies have shown that 50% or more of hospital patients colonized by C. diff are symptomless carriers.

The C. diff bacterium attaches to sugar-containing proteins on the cell surface and produces two exotoxins, toxin A (enterotoxin) and toxin B (cytotoxin), which are pulled further into the cell through invagination of the cell's plasma membrane. Once incorporated into the cell, the amino acid chain of the C. diff toxin divides, causing regulation of the actin cytoskeleton to be impaired, increasing permeability of the intestinal epithelium, as well as increasing apoptosis. C. diff toxin A damages intestinal villous tips and disrupt the brush border membrane, leading to cell erosion and fluid leakage from the damaged intestinal wall. Moreover, stopping the infection does not reliably stop the cycle of recurring infections. C. diff can be a passive resident in a healthy gut biota, but it also forms spores that can remain dormant for years inside the body or on surfaces, re-infecting the body when conditions are right.

C. diff causes an infection in the lining of the gut, resulting in symptoms ranging from diarrhea and colitis to life-threatening inflammation of the colon, often resulting from eradication of the normal gut flora by antibiotics. More serious cases can cause severe damage to the intestines, resulting in the need for surgery. Conditions such as toxic megacolon and colitis are often accompanied with other complicating health problems that can quickly become life threatening. Signs and symptoms of a severe infection include watery diarrhea 10 to 15 times a day, severe abdominal cramping and pain, fever, blood or pus in the stool, nausea, dehydration, loss of appetite, and weight loss.

Other methods are commonly used or prescribed for these infections, depending on the severity, but they have to be carefully selected as they can introduce their own problems. Drugs used to stop diarrhea can be undesirable, as they can worsen the course of C. diff-related pseudomembranous colitis. For example, loperamide, diphenoxylate, and bismuth compounds slow fecal transit time which might result in extended toxin-associated damage. On the other hand, direct mechanisms to reduce C. diff virulence are desirable: (1) reduce the release of the C. diff toxins or ability of the toxins to attach to intestinal epithelial cells, (2) reduce the viability/replication of the C. diff bacterium, and (3) reduce sporulation/spore viability. Likewise, indirect mechanisms of increasing host immunity are desirable, for example, developing a host resistance to infection or reinfection: (4) probiotic modification to the gut microbiome to generate competitive exclusion pressure against C. diff bacteria, (5) improvement of the microbial exclusion function of the mucosal tissues, (6) stimulation of host humoral activation against the pathogen, and (7) physical shielding of vulnerable mucosal tissues against colonization and attack. Moreover, avoiding or reducing the use of antiobiotics can reduce the selective pressure and the current trend toward increasing antibiotic resistance.

Accordingly, one of skill will appreciate having compositions and methods of killing antibiotic-resistant bacteria such as, for example, spore-forming, anaerobic antibiotic-resistant bacteria. In particular, one of skill will appreciate having compositions and methods of killing C. diff. One of skill would appreciate a reliable method of treating C. diff-induced conditions such as, for example, diarrhea and intestinal inflammation, without eradicating normal gut flora or promoting of antibiotic resistance. For at least the reasons discussed above, one of skill will appreciate the teachings provided herein, which include (i) methods of avoiding or reducing the use of antibiotics; (ii) direct mechanisms of reducing C. diff virulence; and (iii) indirect mechanisms of increasing host immunity. Such compositions and methods help, for example, to meet a growing need for effective control of hospital acquired infections (HAIs) resulting from antibiotic-resistant pathogens generally associated with the selective pressure induced by the frequent use of antibiotics. It will be appreciated that the compositions and methods taught herein are an alternative to the use of antibiotics, representing a paradigm shift that reduces clinical symptoms of HAIs without invoking the problematic antibiotic resistance mechanisms that have become such a serious problem to our society.

### SUMMARY

The teachings provided herein are directed to methods and compositions for a broad-spectrum, bactericidal or bacteriostatic treatment of antibiotic-resistant bacteria in animals with a non-toxic agent. In some embodiments, the antibiotic-resistant bacteria are endospores. In some embodiments, the antibiotic-resistant bacteria are anaerobic. In some embodiments, the antibiotic-resistant bacteria are aerobic. In some embodiments the teachings are directed to killing, or at least inhibiting the growth of, or onset of, spore-forming, anaerobic antibiotic-resistant bacteria.

Methods of treating a subject that is hosting an antibiotic-resistant bacteria are provided. Such methods can include administering an effective amount of a formulation to a subject that is hosting an antibiotic-resistant bacteria, the formulation having a water soluble tannin combined with hydrogen peroxide in a pharmaceutically acceptable excipient. The tannin can have a molecular weight ranging from about 170 Daltons to about 4000 Daltons, and the tannin:peroxide weight ratio can range from about 1:1000 to about 10:1. These formulations can at least inhibit the growth of the antibiotic-resistant bacteria in the subject when compared to a second subject in a control group also hosting the antibiotic-resistant bacteria in which the formulation was not administered.

It should be appreciated that gastrointestinal conditions associated with antibiotic-resistant bacteria can be treated using the compositions and methods taught herein. As such, methods of treating a gastrointestinal inflammation in a subject that is hosting the antibiotic-resistant bacteria are provided.

The methods include administering an effective amount of a formulation to a subject that is hosting the antibiotic-resistant bacteria, the formulation produced from a process including combining a water soluble tannin with hydrogen peroxide at a tannin:peroxide weight ratio that ranges from about 1:1000 to about 10:1, the tannin having a molecular weight ranging from about 170 Daltons to about 4000 Daltons. The methods can also include removing free hydrogen peroxide from the combination; and, mixing the combination of the tannin and the hydrogen peroxide with a pharmaceutically acceptable excipient to create the formulation.

Methods of treating diarrhea in a subject that is hosting an antibiotic-resistant bacteria are provided. The method can include administering an effective amount of a composition to a subject that is hosting an antibiotic-resistant bacteria. In such embodiments, the composition can be produced from a process including combining a water soluble, hydrolysable tannin with hydrogen peroxide at a tannin:peroxide weight ratio that ranges from about 1:1000 to about 10:1, the tannin having a molecular weight ranging from about 170 Daltons to about 4000 Daltons

The administering can include selecting a desired concentration of the formulation for the administering; and, the formulation can be used to relieve diarrhea in the subject that is hosting the antibiotic-resistant bacteria, the extent of relief measured as compared to a second subject in a control group also hosting the antibiotic-resistant bacteria in which the formulation was not administered.

Methods of at least inhibiting the onset of, the germination of, or the growth of an antibiotic-resistant bacteria are provided. The methods can include contacting an antibiotic-resistant bacteria with a composition having a water soluble tannin combined with hydrogen peroxide In some embodiments, the tannin can have a molecular weight ranging from about 170 Daltons to about 4000 Daltons; and, in some embodiments, the tannin:peroxide weight ratio can range from about 1:1000 to about 10:1. In these embodiments, the composition can be used to inhibit the growth of the antibiotic-resistant bacteria when compared to a negative control group.

In the embodiments taught herein, the administering of a formulation can include selecting a desired concentration of the formulation for the administering. In some embodiments, for example, the desired concentration can be effect to relieve a discomfort in the subject treated, such as a discomfort in any tissue, for example, a gastrointestinal tissue. In some embodiments, the formulation relieves a gastrointestinal inflammation in the subject that is hosting the antibiotic-resistant bacteria when compared to a second subject in a control group also hosting the antibiotic-resistant bacteria in which the formulation was not administered.

It should be appreciated that the compositions and methods provided herein can at least inhibit the onset of, inhibit the growth of, inhibit the germination of, or kill the antibiotic-resistant bacteria. In some embodiments, the antibiotic-resistant bacteria is Clostridium difficile. In some embodiments, the antibiotic-resistant bacteria is Enterococcus faecalis. In some embodiments, the antibiotic-resistant bacteria is Staphylococcus aureus. And, in some embodiments, the antibiotic-resistant bacteria is Klebsiella pneumoniae.

It should be appreciated that any tannin can be used in the compositions and methods provided herein. In some embodiments, the tannin is gallic acid, epigallic acid, or a combination thereof. In some embodiments, the tannin is an ellagitannin. In some embodiments, the tannin is punicalagin. And, in some embodiments, the tannin is tannic acid.

One of skill reading the teachings that follow will appreciate that the concepts can extend into additional embodiments that go well-beyond a literal reading of the claims, the inventions recited by the claims, and the terms recited in the claims.

### BRIEF DESCRIPTION OF THE FIGURES

FIGS. 1A-1H are photographs of the dry forms of (A) gallic acid (a model polyphenol building block) bound to hydrogen peroxide; (B) gallic acid alone; (C) tannic acid (a model polyphenol) bound to hydrogen peroxide; (D) tannic acid alone; (E) pomegranate husk extract bound to hydrogen peroxide; (F) pomegranate husk extract alone; (G) green tea extract bound to hydrogen peroxide; and (H) green tea extract alone, according to some embodiments.

FIGS. 2A and 2B show that the stability of the hydrogen peroxide in the combination is consistently, substantially greater in an aqueous solution than the stability of the hydrogen peroxide alone in the aqueous solution, according to some embodiments.

FIGS. 3A-3C illustrate an endospore and germination, according to some embodiments.

### DETAILED DESCRIPTION

The teachings provided herein relate to methods and compositions for a broad-spectrum, bactericidal or bacteriostatic treatment of antibiotic-resistant bacteria in animals with a non-toxic agent. In some embodiments, the antibiotic-resistant bacteria are endospores. In some embodiments, the antibiotic-resistant bacteria are anaerobic. In some embodiments, the antibiotic-resistant bacteria are aerobic. In some embodiments the teachings are directed to killing, or at least inhibiting the growth of, or onset of, spore-forming, anaerobic antibiotic-resistant bacteria.

In some embodiments, the compositions and methods taught herein can be used to inhibit the onset of, the growth of, or kill, any endospore. In some embodiments, the compositions and methods provided herein can be used in the bacteriostatic or bactericidal control of carbapenem-resistant Enterobacteriaceae (CRE). Carbapenem-resistant Enterobacteriaceae, are a family of germs that are difficult to treat because they have high levels of resistance to antibiotics. Examples include the Klebsiella (e.g., Klebsiella oxytoca) species, the Citrobacter species (e.g., Citrobacter freundii), and the Escherichia coli (E. coli) species of Enterobacteriaceae. Both are a normal part of the human gut bacteria. Some carbapenem-resistant Enterobacteriaceae (CRE) bacteria have become resistant to most available antibiotics. Infections with these germs are very difficult to treat, and can be deadly. One report cites they can contribute to death in up to 50% of patients who become infected.

As such, methods of treating a subject that is hosting an antibiotic-resistant bacteria are provided. Such methods can include administering an effective amount of a formulation to a subject that is hosting an antibiotic-resistant bacteria, the formulation having a water soluble tannin combined with hydrogen peroxide in a pharmaceutically acceptable excipient. The tannin can have a molecular weight ranging from about 170 Daltons to about 4000 Daltons, and the tannin:peroxide weight ratio can range from about 1:1000 to about 10:1. These formulations can at least inhibit the growth of the antibiotic-resistant bacteria in the subject when compared to a second subject in a control group also hosting the antibiotic-resistant bacteria in which the formulation was not administered.

It should be appreciated that gastrointestinal conditions associated with antibiotic-resistant bacteria can be treated using the compositions and methods taught herein. As such, methods of treating a gastrointestinal inflammation in a subject that is hosting the antibiotic-resistant bacteria are provided.

The methods include administering an effective amount of a formulation to a subject that is hosting the antibiotic-resistant bacteria, the formulation produced from a process including combining a water soluble tannin with hydrogen peroxide at a tannin:peroxide weight ratio that ranges from about 1:1000 to about 10:1, the tannin having a molecular weight ranging from about 170 Daltons to about 4000 Daltons. The methods can also include removing free hydrogen peroxide from the combination; and, mixing the combination of the tannin and the hydrogen peroxide with a pharmaceutically acceptable excipient to create the formulation.

Metallo-beta-lactamase-1 (NDM-1), for example, is an enzyme that makes bacteria resistant to a broad range of beta-lactam antibiotics. These, of course, include the antibiotics of the carbapenem family, which are a mainstay for the treatment of antibiotic-resistant bacterial infections. The gene for NDM-1 is one member of a large gene family that encodes beta-lactamase enzymes called carbapenemases. Bacteria that produce carbapenemases are often referred to in the news media as “superbugs” because infections caused by them are very difficult to treat, usually susceptible to only polymyxins and tigecycline. The most common bacteria that make this enzyme are gram-negative bacteria, such as Escherichia coli and Klebsiella pneumoniae, but the gene for NDM-1 can spread from one strain of bacteria to another by horizontal gene transfer. As such, bacteria can become carbapenem-resistant due to the selective pressure of antibiotic therapies. And, accordingly, some specific types of CRE can be classed by the type of enzymes that make the therapies ineffective: Klebsiella pneumonia carbapenemase (KPC) New Delhi Metallo-beta-lactamase (NDM), the enzymes that breakdown carbapenems and make them ineffective. The numbers of carbapenem-hydrolyzing β-lactamases in members of the family Enterobacteriaceae are increasing in the United States, and the most frequently encountered are the plasmid-encoded Ambler class A Klebsiella pneumoniae carbapenemase (KPC)-type enzymes found in isolates predominantly from the eastern United States, particularly from the New York City region. More recently, the geographical distribution of KPC-producing isolates within the United States has widened to include Pennsylvania, Ohio, Arkansas, Georgia, Colorado, New Mexico, Arizona, and California. KPC-producing Escherichia coli and K. pneumoniae isolates that are thought to have originated outside of the United States have been reported in Israel, Colombia, Greece, and China. KPC was first identified in a K. pneumoniae isolate from North Carolina, and the enzyme has been found the most frequently in K. pneumoniae. In addition, KPC enzymes have been detected in multiple genera and species of the Enterobacteriaceae, including the Salmonella enterica serotype Cubana, K. oxytoca, Enterobacter spp., Citrobacter freundii, E. coli, and Serratia marcescens. A recent report from Colombia also describes KPC-producing isolates of Pseudomonas aeruginosa.

Likewise, in some embodiments, the compositions and methods provided herein can be used in the bacteriostatic or bactericidal control of vancomycin-resistant Enterococci (VRE). The Enterococci are bacteria that are normally present in the human intestines and in the female genital tract. They are also found quite often in our day-to-day environments and can sometimes cause infections. Vancomycin is an antibiotic that is used to treat some drug-resistant infections caused by the Enterococci. In some instances, Enterococci have become resistant to vancomycin and, appropriately, are now called vancomycin-resistant Enterococci (VRE). VRE infections are generally thought to be HAIs, as they typically occur in hospitals.

Likewise, in some embodiments, the compositions and methods provided herein can be used in the bacteriostatic or bactericidal control of methicillin-resistant Staphylococcus aureus (MRSA). MRSA is a type of staph bacteria that is also resistant to the beta-lactam antibiotics, for example, methicillin and other more common antibiotics such as oxacillin, penicillin, and amoxicillin. MRSA infections occur most frequently among patients in healthcare settings, also generally thought to be HAIs.

Likewise, in some embodiments, the compositions and methods provided herein can be used in the bacteriostatic or bactericidal control of C. diff. A case definition of C. diff can include the presence of symptoms (usually diarrhea) and either a stool test result positive for C. diff toxins or findings of pseudomembranous colitis with colonoscopy. There are many strains of C. diff and all are characteristically resistant to most antibiotics. Many antibiotics have been shown to reduce populations of other bacteria, increasing risk of C. diff overgrowth and infection. There are two common antibiotics that are useful against C. diff metronidazole (FLAGYL) and vancomycin (VANCOCIN), both of which are usually taken orally. In both cases, significant side effects including gastric distress are common. Metronidazole is the preferred antibiotic treatment for mild cases of C. diff but increasing resistance is making it less effective every year. Vancomycin is usually reserved for moderate to severe infections. A few newer antibiotics, such as rifaximin (RIFAGUT) have shown promising results in some cases. Sometimes multiple courses of these antibiotics are used to try to control recurring C. diff. infections. As such, antibiotics can be used, in some embodiments, in combination with the compositions taught herein in the methods taught herein.

As such, methods of treating diarrhea in a subject that is hosting an antibiotic-resistant bacteria are provided. The method can include administering an effective amount of a composition to a subject that is hosting an antibiotic-resistant bacteria. In such embodiments, the composition can be produced from a process including combining a water soluble, hydrolysable tannin with hydrogen peroxide at a tannin:peroxide weight ratio that ranges from about 1:1000 to about 10:1, the tannin having a molecular weight ranging from about 170 Daltons to about 4000 Daltons

The administering can include selecting a desired concentration of the formulation for the administering; and, the formulation can be used to relieve diarrhea in the subject that is hosting the antibiotic-resistant bacteria, the extent of relief measured as compared to a second subject in a control group also hosting the antibiotic-resistant bacteria in which the formulation was not administered.

To provide a desired therapeutic relief, the compositions can be directed to act on tissues at a particular target site, which can be gastrointestinal tissue, in some embodiments. In some embodiments, the compositions can be directed to act on reproductive tract tissue, urinary tract tissue, nasopharyx tissue, esophageal tissue, sinus tissue, or other mucosal tissues. The term “target site” can be used to refer to a select location at which the composition acts to provide a therapeutic effect, or treatment as described herein. In some embodiments, the target site can be a tissue of any organ in which inhibiting the growth of an antibiotic-resistant bacteria is desirable. In some embodiments, the target can include any site of action in which the phenolic compound can be site-activated by an oxidoreductase enzyme that is available at the site. The oxidoreductase enzyme can be produced endogeneously by a tissue at a target site, produced endogeneously by a microbe, introduced exogenously to the target site, include more than one enzyme, co-enzyme, catalyst, or cofactor, or a combination thereof. In some embodiments, the compositions can be used on non-mucosal tissue, such as dermal tissue. In fact, in some embodiments, the compositions can be used on medical devices or other surfaces to inhibit, or prevent, the growth of bacteria and, most importantly, antibiotic-resistant bacteria.

One of skill will appreciate that an endospore can tolerate extreme environmental conditions and remain viable for a very long time, often many years, after which the endospore can absorb water, swell, and release a new bacterium from the endospore. As such, that person of skill will appreciate that methods of at least inhibiting the onset of, the germination of, or the growth of an antibiotic-resistant bacteria are provided. The methods can include contacting an antibiotic-resistant bacteria with a composition having a water soluble tannin combined with hydrogen peroxide. In some embodiments, the tannin can have a molecular weight ranging from about 170 Daltons to about 4000 Daltons; and, in some embodiments, the tannin:peroxide weight ratio can range from about 1:1000 to about 10:1. In these embodiments, the composition can be used to inhibit the growth of the antibiotic-resistant bacteria when compared to a negative control group.

In the embodiments taught herein, the administering of a formulation can include selecting a desired concentration of the formulation for the administering. In some embodiments, for example, the desired concentration can be effect to relieve a discomfort in the subject treated, such as a discomfort in any tissue, for example, a gastrointestinal tissue. In some embodiments, the formulation relieves a gastrointestinal inflammation in the subject that is hosting the antibiotic-resistant bacteria when compared to a second subject in a control group also hosting the antibiotic-resistant bacteria in which the formulation was not administered.

It should be appreciated that the compositions and methods provided herein can at least inhibit the onset of, inhibit the growth of, inhibit the germination of, or kill the antibiotic-resistant bacteria. In some embodiments, the antibiotic-resistant bacteria is Clostridium difficile. In some embodiments, the antibiotic-resistant bacteria is Enterococcus faecalis. In some embodiments, the antibiotic-resistant bacteria is Staphylococcus aureus. And, in some embodiments, the antibiotic-resistant bacteria is Klebsiella pneumoniae.

It should be appreciated that any tannin can be used in the compositions and methods provided herein. In some embodiments, the tannin is gallic acid, epigallic acid, or a combination thereof. In some embodiments, the tannin is an ellagitannin. In some embodiments, the tannin is punicalagin. And, in some embodiments, the tannin is tannic acid.

Without intending to be bound by any theory or mechanism of action, the methods and formulations taught herein can include phenolics, for example, polyphenols. The methods and formulations taught herein can combine an agent, such as a tannin, with a reactive oxygen species to form a composition that is deliverable as a stable, or substantially stable, system. In some embodiments, the formulations include a combination of components having an association that offers a stability and activity, both of which are offered by neither component alone. Such formulations can be delivered to a target site, for example, in a polar solution such as water or an alcohol. In some embodiments, at least a substantial amount of the hydrogen peroxide can remain bound, or otherwise associated with, and thus stable or substantially stable, with the agent. Moreover, in some embodiments, the formulation contains no, or substantially no, unbound hydrogen peroxide. The teachings also include a pharmaceutical formulation comprising the combinations taught herein and a pharmaceutically acceptable excipient.

The terms “composition,” “compound,” “binding system,” “binding pair,” “formulation,” “combination,” and “system” can be used interchangeably in some embodiments and, it should be appreciated that a “formulation” can comprise a composition, compound, binding system, binding pair, or system presented herein. Likewise, in some embodiments, the compositions taught herein can also be referred to as an “agent,” a “bioactive agent,” or a “supplement” whether alone, in a pharmaceutically acceptable composition or formulation, and whether in a liquid or dry form. Moreover, the term “bioactivity” can refer to a treatment that occurs through the use of the compositions provided herein. One of skill will appreciate that the term “bind,” “binding,” “bound,” “attached,” “connected,” “chemically connected,” “chemically attached,” “combined,” or “associated” can be used interchangeably, in some embodiments. Such terms, for example, can be used to refer to any association between the agent and reactive oxygen species that has resulted in an increased stability and/or sustained activity of the composition or components in the compositions. For example, the terms can be used to describe a chemical bonding mechanism known to one of skill, such as covalent, ionic, dipole-dipole interactions, London dispersion forces, and hydrogen bonding, for example. In some embodiments, the formulation can comprise a phenolic compound sharing hydrogen bonds with a reactive oxygen species, for example, such as hydrogen peroxide. In some embodiments, the agent can comprise a polyphenol that covalently binds to an amino acid or polyol.

One of skill will appreciate that the compositions should remain stable, or at least substantially stable, until useful or activated, and this can relate to a measure of time. Such a measure of time can include a shelf life, or a time between creation of the composition and administration of the composition, or some combination thereof. In some embodiments, the composition is stable, or substantially stable, when usable as intended within a reasonable amount of time. In some embodiments, the composition should be usable within a reasonable time from the making of the composition to the administration of the composition and, in some embodiments, the composition should have a reasonable commercial shelf life.

The activity of the composition can include, for example, oxidation potential, ability to precipitate proteins, ability to inhibit microbial activity, or ability to inhibit antibody activity. As such, in some embodiments, the loss of activity can be measured by comparing it's ability to precipitate proteins after making the composition to the time of administration, and this can include a reasonable shelf life. In some embodiments, the loss can be measured by comparing it's ability to inhibit microbial activity after making the composition to the time of administration, and this can include a reasonable shelf life. In some embodiments, the loss can be measured by comparing it's ability to inhibit antibody activity after making the composition to the time of administration, and this can include a reasonable shelf life.

The composition can be considered as “stable” if it loses less than 20% of it's original activity. In some embodiments, the composition can be considered as stable if it loses less than 10%, 5%, 3%, 2%, or 1% of it's original activity. The composition can be considered as “substantially stable” if it loses greater than about 20% of it's activity, as long as the composition can perform it's intended use to a reasonable degree of efficacy. The loss of activity of the composition can be measured, for example, by comparing it's oxidation potential after making the composition to the time of administration, and this can include a reasonable shelf life, in some embodiments. In some embodiments, the composition can be considered as substantially stable if it loses greater than about 12%, about 15%, about 25%, about 35%, about 45%, about 50%, about 60%, 70% or even about 90% of it's activity. The time to compare the oxidation potential for a measure of stability can range from about 30 minutes to about one hour, from about one hour to about 12 hours, from about 12 hours to about 1 day, from about one day to about one week, from about 1 week to about 1 month, from about 1 month to about 3 months, from about 1 month to a year, from 3 months to a year, from 3 months to 2 years, from 3 months to 3 years, greater than 3 months, greater than 6 months, greater than one year, or any time or range of times therein, stated in increments of one hour.

One of skill will appreciate that the phenolic compound used in the compositions can be any phenolic compound that functions consistent with the teachings provided herein, and there are at least several thousand such phenolic compounds known to those of skill that can be expected to function as desired. As such, the teachings provided herein can only include examples of the general concepts rather than a comprehensive listing of all possibilities and permutations of the systems that are enabled by the teachings.

It is to be appreciated that the phenols include polyphenols. As such, the agent can be a phenol that is not a polyphenol. Moreover, the polyphenol component can comprise a single polyphenol component, a limited mixture of polyphenol components combined in a desired ratio, or a whole extract of a plant tissue which is a complex mixture of polyphenol components, in some embodiments.

A limited mixture can include a preselected ratio of 2, 3, 4, 5, 6, 7, 8, 9, or 10 phenol components, in some embodiments. In some embodiments, the limited mixture can include a preselected ratio of 11, 12, 13, 14, 15, 16, 17, 18, 19, or 20 phenol components. In some embodiments, the polyphenol comprises a tannin. In some embodiments, the polyphenol comprises a hydrolysable tannin, a condensed tannin, or a combination of a hydrolysable tannin and a condensed tannin. In some embodiments, the polyphenol can comprise a pseudotannin selected, for example, from the group consisting of gallic acid, which can be found in an extract of a rhubarb plant tissue, for example; flavan-3-ols or catechins, which can be found in an extract of acacia, catechu, cocoa, or guarana, for example; chlorogenic acid, which can be found in coffee, or mate; or, ipecacuanhic acid, which can be found in carapichea ipecacuanha, for example. As such, it should be appreciated that, in some embodiments, the polyphenol component can comprise a flavanol or a catechin. Moreover, the polyphenol can comprises gallic acid, epigallic acid, or a combination thereof, in some embodiments. In some embodiments, the agent can be tannic acid.

In some embodiments, the phenolic compound has at least one aryl group, or arene moiety, and at least two polar aromatic groups, such as aromatic hydroxyl groups. In some embodiments, the polar aromatic groups can be, for example, hydroxyl, amine, amide, acyl, carboxy, or carbonyl. In some embodiments, the phenolic compound has at least two aryl groups, and at least two hydroxyl groups. In some embodiments, the phenolic compounds can be naturally occurring, such as from a plant or other natural product. And, in some embodiments, the phenolic compounds can be synthetically or semi-synthetically produced. The compounds can be simple monomers, oligomers, or polymers. The polymers can be in the class of polyphenols or polymeric phenols, where one of skill will understand that the general difference is typically that polyphenols generally do not have a repeating unit, whereas polymeric phenols do. There are exceptions, however, such that groups of polyphenols and polymeric phenols can overlap. In most embodiments, the phenolic compound used in the binding system can be any phenolic compound taught herein, or any prodrugs, codrugs, metabolites, analogs, homologues, congeners, derivatives, salts, solvates, and combinations thereof.

In some embodiments, the phenolic compounds bind to hydrogen peroxide to form a binding pair and, in some embodiments, the binding pair remains stable, or substantial stable in water. In some embodiments, the binding pair remains stable, or substantial stable in an alcohol. And, in some embodiments, the binding pair remains stable, or substantial stable, in a polar solvent such as, for example, a saline solution, an aqueous emulsion, a hydrogel, and the like.

In some embodiments, the phenolic compounds are polyphenols having molecular weights ranging from about 170 to about 4000 Daltons, having from about 12 to about 16 phenolic hydroxyl groups, and having from about five to about seven aromatic rings, for every about 1000 Daltons in molecular weight. In some embodiments, the phenolic compounds function to precipitate alkaloids and proteins. In some embodiments, the phenolic compounds can bind to cellular receptors, amino acids, peptides, oligopeptides, polyols, saccharides, or combinations thereof. In some embodiments, the phenolic compounds have at least from about 1 to about 20 polyhydroxylated phenolic units and have at least moderate water solubility.

The term “solubility” can refer to a concentration of a solute in a solvent, for example, the phenolic compound in water. The concentration can be expressed by mass, for example, mg of the phenolic compound per kg of water at ambient temperature and pressure. This ratio of mg/kg can be used interchangeably with ppm, and ng/kg can be used interchangeably with ppb. In some embodiments, the solubility of the phenolic compound can be higher than about 500,000 ppm or less than about 1 ppm. In some embodiments, the solubility of the phenolic compound range from about 10 ppb to about 500,000 ppm, from about 100 ppb to about 250,000 ppm, from about 1 ppm to about 100,000 ppm, from about 10 ppm to about 50,000 ppm, from about 50 ppm to about 25,000 ppm, from about 100 ppm to about 10,000 ppm, from about 100 ppm to about 100,000 ppm, from about 200 ppm to about 100,000 ppm, from about 250 ppm to about 50,000 ppm, from about 500 ppm to about 25,000 ppm from about 250 ppm to about 10,000 ppm, or any range therein. In some embodiments, the solubility can range from about 1 g/L to about 10,000 g/L, from about 5 g/L to about 5000 g/L, from about 10 g/L to about 3000 g/L, from about 20 g/L to about 2000 g/L, from about 50 g/L to about 1000, g/L, from about 100 g/L to about 500 g/L, or any range therein. For purposes of the teachings provided herein, a compound can be considered to have a low solubility if the solubility is less than about 50 g/L, a moderate solubility if the solublity ranges from about 50 g/L to about 1000 g/L, and a high solubility if the solubility is above about 1000 g/L. In some embodiments, the phenolic compound can have a low solubility. In some embodiments, the phenolic compound can have a moderate solubility. And, in some embodiments, the phenolic compound can have a high solubility.

One of skill will appreciate that the phenolic compounds can still be useful at low solubilities in cases where the solubility is too low to form a true solution. In some embodiments the phenolic compounds can be ground into particles to form a colloidal mixture or suspension that will function consistent with the teachings provided herein. As such, liquid formulations include colloids and suspensions in some embodiments. The formulations can be a dispersed phase mixture in the form of colloidal aerosols, colloidal emulsions, colloidal foams, colloidal dispersions, or hydrosols. In some embodiments, the liquid formulation can include particles having sizes ranging, for example, from about 5 nm to about 200 nm, from about 5 nm to about 500 nm, from about 5 nm to about 750 nm, from about 50 nm to about 1 um. In some embodiments, the liquid formulations can be suspensions, in which the particle size range from about 1 um to about 10 um, from about 1 um to about 7 um, from about 1 um to about 5 um, or any range therein. In some embodiments, the liquid formulation can include particles having sizes ranging from about 1 nm to about 10 um.

The functionality of a phenolic compound in the teachings herein can, for at least the reason of solubility, depend on molecular weight, alone or in addition to other factors discussed herein such as, for example, extent of hydroxylation, presence and location of ketone or quinine groups, and the presence of other functional groups. In some embodiments, the molecular weights of the phenolic compounds can range from about 110 Daltons to about 40,000 Daltons. In some embodiments, the molecular weights of the phenolic compounds can range from about 200 Daltons to about 20,000 Daltons, from about 300 Daltons to about 30,000 Daltons, from about 400 Daltons to about 40,000 Daltons, from about 500 Daltons to about 10,000 Daltons, from about 1000 Daltons to about 5,000 Daltons, from about 170 Daltons to about 4000 Daltons, from about 350 Daltons to about 4,000 Daltons, from about 300 Daltons to about 3,000 Daltons, from about 110 Daltons to about 2,000 Daltons, from about 200 to about 5000 Daltons, or any range or molecular weight therein in increments of 10 Daltons.

In some embodiments, the ratio of aromatic rings to molecular weight of the phenolic compounds can range from about five to about seven aromatic rings for every about 1000 Daltons. In some embodiments, the ratio of aromatic rings to molecular weight of the phenolic compounds can range from about 2 to about 10 aromatic rings for every about 1000 Daltons, from about 3 to about 9 aromatic rings for every about 1000 Daltons, from about 4 to about 8 aromatic rings for every about 1000 Daltons, from about 5 to about 7 aromatic rings for every about 1000 Daltons, from about 1 to about 5 for every about 500 Daltons, from about 1 to about 4 for every about 500 Daltons, from about 1 to about 3 for every about 500 Daltons, from about 2 to about 4 for every about 500 Daltons, or any amount or range therein in increments of 1 ring.

One of skill will appreciate that, in some embodiments the phenolic compounds can have, or be synthesized or otherwise designed to contain functional groups that are capable of releasably bonding to a reactive oxygen species, in a stable or substantially stable form, until either consumed or released upon bioactivation at a target site. In some embodiments, a releasable bond can include any bond other than a covalent bond. In some embodiments, a releasable bond is a hydrogen bond. As such, the phenolic compounds should be capable of forming, for example, a hydrogen bond with a reactive oxygen species upon such bioactivation. In some embodiments, the phenolic compound shares hydrogen bonding with hydrogen peroxide and is released through a bioactivation that occurs when the binding pair comes into contact with an oxidoreductase enzyme or other reducing agent. In some embodiments, the phenolic compound can have functional groups that comprise acyl, amido, amino, carbonyl, carboxyl, hydroxyl, or peroxyl functionality. In some embodiments, the hydrogen bond between the reactive oxygen species and the phenolic compound can include any hydrogen donor and any hydrogen acceptor having an available lone pair of electrons. In some embodiments, the hydrogen acceptor can include, for example a N, O, or F atom, or a combination thereof. In some embodiments, the phenolic compound can have such a functionality, can be derivatized to have such a functionality, can be linked to another compound having such a functionality, can be placed in a carrier having such a functionality, or some combination thereof.

In some embodiments, phenolic compounds can include simple phenols, such as those containing 6 carbons, a C6 structure, and 1 phenolic cycle, such as the benzene alcohols, examples of which include phenol, benzene diols and it's isomers such as catechol, and the benzenetriols. In some embodiments, phenolic compounds can include phenolic acids and aldehydes, such as those containing 7 carbons, a C6-C1 structure, and 1 phenolic cycle, examples of which include gallic acid and salicylic acids. In some embodiments, phenolic compounds can include, for example, tyrosine derivatives, and phenylacetic acids, such as those containing 8 carbons, a C6-C2 structure, and 1 phenolic cycle, examples of which include 3-acetyl-6-methoxybenzaldehyde, tyrosol, and p-hydroxyphenylacetic acid. In some embodiments, phenolic compounds can include hydroxycinnamic acids, phenylpropenes, chromones, such as those containing 9 carbons, a C6-C3 structure, and 1 phenolic cycle, examples of which include caffeic acid, ferulic acids, myristicin, eugenol, umbelliferone, aesculetin, bergenon, and eugenin. In some embodiments, phenolic compounds can include naphthoquinones, such as those containing 10 carbons, a C6-C4 structure, and 1 phenolic cycle, examples of which include juglone and plumbagin. In some embodiments, phenolic compounds can include xanthonoids, such as those containing 13 carbons, a C6-C1-C6 structure, and 2 phenolic cycles, examples of which include mangiferin. In some embodiments, phenolic compounds can include stilbenoids, and anthraquinones, such as those containing 14 carbons, a C6-C2-C6 structure, and 2 phenolic cycles, examples of which include resveratrol and emodin. In some embodiments, phenolic compounds can include chalconoids, flavonoids, isoflavonoids, and neoflavonoids, such as those containing 15 carbons, a C6-C3-C6 structure, and 2 phenolic cycles, examples of which include quercetin, myricetin, luteolin, cyanidin, and genistein. In some embodiments, phenolic compounds can include lignans and neolignans, such as those containing 18 carbons, a C6-C3-C6 structure, and 2 phenolic cycles, examples of which include pinoresinol and eusiderin. In some embodiments, phenolic compounds can include biflavonoids, such as those containing 30 carbons, a (C6-C3-C6)₂ structure, and 4 phenolic cycles, examples of which include amentoflavone. In some embodiments, phenolic compounds can include polyphenols, polyphenolic proteins, lignins, and catechol melanins, such as those containing >30 carbons. In these embodiments, the phenolic compounds can have, for example, a (C6-C3)ₙ structure, a (C6)ₙ structure, a (C6-C3-C6)ₙ structure, or some combination thereof, as well as greater than about 12 phenolic cycles. Examples of such embodiments can include, for example, the flavolans, in the class of condensed tannins.

In some embodiments, the phenolic compounds are natural phenols that can be enzymatically polymerized. Derivatives of natural phenols can also be used in some embodiments. These embodiments can include phenolic compounds having less than 12 phenolic groups, such that they can range from monophenols to oligophenols. In some embodiments, the natural phenols are found in plants, have an antioxidant activity, or a combination thereof. Examples of the natural phenols include, for example, catechol- and resorcinol-types (benzenediols) with two phenolic hydroxy groups, and pyrogallol- and phloroglucinol-types (benzenetriols) with three hydroxy groups. Natural phenols may have heteroatom substituents other than hydroxyl groups, ether and ester linkages, carboxylic acid derivatives, or some combination thereof. In some embodiments, the natural phenols include natural phenol drugs and their derivatives. Examples of such drugs include, but are not limited to, anthraquinone drugs, flavone drugs, and flavonol drugs. Examples of anthraquinone drugs include, but are not limited to, aloe emodin, aquayamycin, and diacerein. Examples of flavone drugs include, but are not limited to, ansoxetine and hidrosmin. Examples of flavonol drugs include, but are not limited to, monoxerutin and troxerutin.

In some embodiments, the phenolic compound is a tannin, a polyphenolic phenylpropanoid, or a combination thereof. In some embodiments, the tannin is a hydrolysable tannin, a condensed tannin, or a combination thereof. Hydrolysable tannins can be found, for example, in chinese gall, which is almost pure in that it has no or substantially no condensed tannins. Condensed tannins can be found, for example, in green tea leaf, which is also almost pure in that it has no or substantially no hydrolysable tannins.

Examples of hydrolysable tannin can include gallotannic acids, quercitannic acids, ellagitannins, gallotannin, pentagalloyl glucose, galloylquinic acid, galloyl-shikimic acid, punicalagin, and punicalin. In some embodiments, the hydrolysable tannin is a gallotannin or ellagitannin, and isomers thereof, such as isomers that can precipitate protein. Examples of gallotannins include the gallic acid esters of glucose in tannic acid (C₇₆H₅₂O₄₆) and pentagalloyl glucose (PGG), and isomers thereof, such as the isomers of PGG that function to precipitate proteins. Examples of an ellagitannin can include castalin, punicalagin, and punicalin. In some embodiments, the agent can include punicalagin, punicalin, or a combination thereof. The combination can be a ratio of punicaligin:punicalin ranging from about 1:100 to about 100:1, from about 1:75 to about 75:1, from about 1:50 to about 50:1, from about 1:25 to about 25:1, from about 1:10 to about 10:1, from about 1:5 to about 5:1, from about 1:3 to about 3:1, from about 1:2 to about 2:1, from about 1:1.5 to about 1.5:1, or any range therein. In some embodiments, the tannin is a gallic acid ester having a molecular weight ranging from about 500 Daltons to about 3000 Daltons. In some embodiments, the tannin is a proanthocyanidin having a molecular weight of up to about 20,000 Daltons. In some embodiments, the hydrolysable tannins are derivatives of gallic acid and characterized by a glucose, quinic acid or shikimic acid core with its hydroxyl groups partially or totally esterified with gallic acid or ellagic acid groups. The compounds can have 3 to 12 galloyl residues but may be further oxidatively cross-linked and complex. Hydrolysable tannins can be readily synthesized, for example, to obtain a phenolic compound with a high number of polar functional groups that form multiple, stable hydrogen bonds between the tannin and hydrogen peroxide in the binding system.

It should be appreciated that, while hydrolysable tannins and most condensed tannins are water soluble, some very large condensed tannins are insoluble. In some embodiments, the phenolic compound can comprise a hydrolysable tannin such as, for example, burkinabin C, castalagin, castalin, casuarictin, chebulagic acid, chebulinic acid, corilagin, digallic acid, ellagitannin, gallagic acid, gallotannin, glucogallin, grandinin, hexahydroxydiphenic acid, pentagalloyl glucose, punicalagin alpha, punicalagins, raspberry ellagitannin, roburin A, stenophyllanin A, stenophyllanin A, tannate, tannic acid, tellimagrandin II, terflavin B, or 3,4,5-tri-O-galloylquinic acid.

In some embodiments, the phenolic compound can be a flavonoid which includes several thousand natural phenol compounds. Examples of the flavonoids include the flavonols, flavones, flavan-3ol (catechins), flavanones, anthocyanidins, isoflavonoids, and hybrids of any combination of these compounds. In some embodiments, the phenolic compounds are the hydrolysable tannins such as, for example, gallic acid. In some embodiments, the phenolic compounds are the lignins such as, for example, cinnamic acid. In some embodiments, the phenolic units can be dimerized or further polymerized to form any of a variety of hybrids. For example, ellagic acid is a dimer of gallic acid and forms the class of ellagitannins, or a catechin and a gallocatechin can combine to form theaflavin or the large class of thearubigins found in tea. In another example, a flavonoid and a lignan can combine to form a hybrid, such a flavonolignans.

In some embodiments, the phenolic compound can be a flavan-3ol. Examples include the catechins and the catechin gallates, where the catechin gallates are gallic acid esters of the catechins. In some embodiments, the phenolic compound is a catechin or epicatechin compound (the cis- or trans-isomers). In some embodiments, the phenolic compound is (−)-epicatechin or (+)-catechin. In some embodiments, the phenolic compound is epigallocatechin (EGC) or gallocatechin (EC). In some embodiments, the phenolic compound is a catechin gallate, such as epigallocatechin gallate (EGCG)

In some embodiments, the phenolic compound can be selected from the group of flavones consisting of apigenin, luteolin, tangeritin, flavonols, isorhamnetin, kaempferol, myricetin (e.g., extractable from walnuts), proanthocyanidins or condensed tannins, and quercetin and related phenolic compounds, such as rutin.

In some embodiments, the phenolic compound can be selected from the group of flavanones consisting of eriodictyol, hesperetin (metabolizes to hesperidin), and naringenin (metabolized from naringin).

In some embodiments, the phenolic compound can be selected from the group of flavanols consisting of catechin, gallocatechin and their corresponding gallate esters, epicatechin, epigallocatechin and their corresponding gallate esters, theaflavin and its gallate esters, thearubigins, isoflavone phytoestrogens (found primarily in soy, peanuts, and other members of the Fabaceae family), daidzein, genistein, glycitein, stilbenoids, resveratrol (found in the skins of dark-colored grapes, and concentrated in red wine), pterostilbene (methoxylated analogue of resveratrol, abundant in Vaccinium berries), anthocyanins, cyanidin, delphinidin, malvidin, pelargonidin, peonidin, and petunidin. And, In some embodiments, the phenolic compound can be ubiquinol an electron-rich (reduced) form of coenzyme Q10.

In some embodiments, the phenolic compound can be selected from the group of carotenoid terpenoid consisting of alpha-carotene, astaxanthin (found naturally in red algae and animals higher in the marine food chain, a red pigment familiarly recognized in crustacean shells and salmon flesh/roe), beta-carotene (found in high concentrations in butternut squash, carrots, orange bell peppers, pumpkins, and sweet potatoes), canthaxanthin, lutein (found in high concentration in spinach, kiwifruit and red peppers), lycopene (found in high concentration in ripe red tomatoes and watermelons) and zeaxanthin (the main pigment found in yellow corn, also abundant in kiwifruit).

In some embodiments, the phenolic compound can be selected from the group of phenolic acids and their esters consisting of chicoric acid (another caffeic acid derivative, is found only in the medicinal herb echinacea purpurea), chlorogenic acid (found in high concentration in coffee (more concentrated in robusta than arabica beans, blueberries and tomatoes, and produced from esterification of caffeic acid), cinnamic acid and its derivatives, such as ferulic acid (found in seeds of plants such as in brown rice, whole wheat and oats, as well as in coffee, apple, artichoke, peanut, orange and pineapple), ellagic acid (found in high concentration in raspberry and strawberry, and in ester form in red wine tannins), ellagitannins (hydrolysable tannin polymer formed when ellagic acid, a polyphenol monomer, esterifies and binds with the hydroxyl group of a polyol carbohydrate such as glucose), gallic acid (found in gallnuts, sumac, witch hazel, tea leaves, oak bark, and many other plants), gallotannins (hydrolysable tannin polymer formed when gallic acid, a polyphenol monomer, esterifies and binds with the hydroxyl group of a polyol carbohydrate such as glucose), rosmarinic acid (found in high concentration in rosemary, oregano, lemon balm, sage, and marjoram), and salicylic acid (found in most vegetables, fruits, and herbs; but most abundantly in the bark of willow trees, from where it was extracted for use in the early manufacture of aspirin).

In some embodiments, the phenolic compound can be selected from the group of nonflavonoid phenolics consisting of curcumin (has low bioavailability, because, much of it is excreted through glucuronidation, but bioavailability can be substantially enhanced by solubilization in a lipid (oil or lecithin), heat, addition of piperine, or through nanoparticularization, flavonolignans, for example, silymarin which is a mixture of flavonolignans extracted from milk thistle), eugenol and xanthones (mangosteen, for example, is purported to contain a large variety of xanthones, some of which, like mangostin are believed to be present only in the inedible shell).

In some embodiments, the phenolic compound can have a low molecular weight (less than about 400 Daltons), selected from the group consisting of caffeic acid, gentisic acid, protocatechuic acid, phenylacetic acid, gallic acid, phloroglucinol carboxylic acid, and derivatives thereof. Such compounds can form a sufficiently soluble binding pair, and their relatively high hydroxyl group to molecular weight ratio creates favorable conditions for obtaining the intermolecular hydrogen bonds desired for the binding systems.

In some embodiments, the phenolic compounds can be from a natural extract, such as an extract of a plant or other natural product. See, for example, U.S. Published Patent Application Nos. 20100158885 and 20110070198, each of which is hereby incorporated by reference herein in its entirety. Those skilled in the art of such extracts will understand that extracts of plant materials are not typically pure in one type of phenolic compound. Plant tannin extracts, for example, typically comprise heterogenous mixtures and derivatives of the above classes.

One of skill will appreciate, given the teachings provided herein, that the polyphenol can be combined with the reactive oxygen species as a component of a water and/or alcohol extract of a plant tissue, the alcohol process comprising, for example, a methanol, ethanol, propanol, 2-propanol, butanol, t-butanol, and the like, and sometimes using a second agent such as 0.1-1.0% dithiothreitol (DTT). In some embodiments, the extraction process can include a mixture of water and alcohol, or a stepwise extraction of water and alcohol in series in any combination.

In some embodiments, the plant tissue can comprise a tannin or a pseudotannin. In some embodiments, the phenolic compound is extracted from a whole or partial plant tissue selected from the group consisting of seeds and fruits; ovaries; juice; pulp; galls; husks; bark; stems; leaves; flowers; sheaths; hulls; sprouts; bulbs; hips; tubers; roots of grains; grasses; legumes; trees; vegetables; medicinal herbs; tea leaves; algaes; marine plants; and, forages. One of skill will appreciate that the type and content of phenolic compound obtained can be expected to vary with the species, season, geographical location, cultivation, and storage. Examples of plant tissues include, but are not limited to, plant tissues from the species of Aloe, Pachycereus, and Opuntia. Other examples can include, but are not limited to, Agavaceae, Cactaceae, Poaceae, Theaceae, Leguminosae, and Lythraceae. In some embodiments, the plant tissues can be selected from the group consisting of pomegranate husk, aloe vera leaves, and green tea leaves. Other examples of plant tissues can include, but are not limited to Aloe (Aloe vera), Angelica (Angelica archangelica), Barberry (Berberis vulgaris) Root Bark, Bilberry (Vaccinium myrtillus), Calendula (Calendula officinalis), Cramp bark (Viburnum opulus), Eleutherococcus root (Eleutherococcus senticosus), Kidney wood (Eysenhardtia orththocarpa), Mimosa tenuiflora, Papaya (Carica papaya) leaves, Pau D' Arco (Tabebuia avellanedae), Sassafras albidum root bark, Saw Palmatto (Serenoa repens), St John's wort (Hypericum perforatum), Valerian (Valeriana officinalis), Apple (Malus domestica), Grape (Vitis vinifera), Echinacea purpurea, Grape seed extract, and Blueberry (Vaccinium corymbosum). In some embodiments, the plant tissues are selected from the group consisting of barley germ, green tea leaves, aloe vera leaves, mung beans, carrot, cereal grains, seeds, buds, and sprouts.

Likewise, one of skill will appreciate that there are numerous reactive oxygen species that can be used in the systems taught herein, as long as the reactive oxygen species function consistent with such teachings. Hydrogen peroxide, and precursors of hydrogen peroxide, are merely examples. In some embodiments, the phenolic compounds in the compositions (i) have phenolic hydroxyl groups that are oxidizable in the presence of a reactive oxygen species and an oxidoreductase enzyme, and (ii) are soluble in a polar liquid, such as water or an alcohol, for example, or at least moderately soluble. The phenolic compounds should also be (iii) non-toxic to a subject upon administration. And, in some embodiments, the phenolic compounds should also (iv) crosslink or polymerize with itself or other phenolic compounds in the compositions taught herein.

The reactive oxygen species can be any such species known to one of skill to have the ability to combine with the polyphenol as a composition for the uses taught herein. For example, the reactive oxygen species can include, but is not limited to, the reactive oxygen species includes a component selected from the group consisting of hydrogen peroxide, superoxide anion, singlet oxygen, and a hydroxyl radical. In some embodiments, the reactive oxygen species comprises hydrogen peroxide. And, in some embodiments, the hydrogen peroxide can be combined with the tannin at a tannin:peroxide weight ratio that ranges from about 1:1000 to about 100:1. In some embodiments, the hydrogen peroxide can be combined with the tannin at a tannin:peroxide weight ratio that ranges from about 1:1000 to about 10:1. In some embodiments, the weight ratio of the tannin:peroxide ranges from about 1:1 to about 1:50. In some embodiments, the weight ratio of the tannin:peroxide is about 1:1, about 1:2, about 1:3, about 1:4, about 1:5, about 1:6, about 1:7, about 1:8, about 1:9, about 1:10, about 1:15, about 1:20, about 1:25, about 1:30, about 1:40, about 1:50, or any ratio therein. In some embodiments, the exogeneous reactive oxygen species can be generated, as hydrogen peroxide for example, from a solid hydrogen peroxide generating material selected from the group consisting of sodium percarbonate, potassium percarbonate, a carbamide peroxide, and urea peroxide.

In some embodiments, the reactive oxygen species is hydrogen peroxide or materials that release or generate hydrogen peroxide including, but not limited to, hydration of adducts of hydrogen peroxide such as carbamide peroxide, magnesium peroxide, and sodium percarbonate; amino perhydrates; superoxide dismutase decomposition of ozone, superoxides or superoxide salts; glucose oxidase and glucose, aqueous dilution of honey; H₂O₂ production by lactobacillus; catalytic quinone hydrogenation; superoxides; and, superoxide dismutase. In some embodiments, the reactive oxygen species can include peroxide ion, organic peroxides, organic hydroperoxides, peracid superoxides, dioxygenyls, ozone, and ozonides. hydrogen peroxide or materials that generate hydrogen peroxide can be obtained or derived synthetically or from plant tissues or combinations of plant tissues.

Enzymes can activate the compositions for the methods taught herein, and the systems for the methods of treatment can be designed accordingly. And, generally speaking, one of skill will appreciate that there are a wide variety of enzymes are possible and can be target site dependent. Generally, the enzymes fall into the classes of oxidoreductases. As such, there are several enzymes and isozymes that will be present at a target site and capable of bioactivating the binding systems. In some embodiments, the oxidoreductases can be categorized into about 22 classes, and the selectivity of the bioactivation of the binding system at a target site depends, at least in part, on the selectivity of the oxidoreductase at the target site. In some embodiments, the oxidoreductase can include those oxidoreductases that act on the CH—OH group of donors (alcohol oxidoreductases, for example; EC Number class 1.1). In some embodiments, the oxidoreductase can include those oxidoreductases that act on diphenols and related substances as donors (catechol oxidase, for example, EC Number class 1.10). In some embodiments, the oxidoreductase can include those oxidoreductases that act on peroxide as an acceptor (peroxidases, such as horseradish peroxidase and catalase; EC Number class 1.11). In some embodiments, the oxidoreductase can include those oxidoreductases that act on phenols as an acceptor (tyrosinases, for example; EC Number class 1.14). Examples of other useful enzymes for the teachings provided herein include, but are not limited to, glutathione peroxidase 1 and 4 (in many mammalian tissues), glutathione peroxidase 2 (in intestinal and extracellular mammalian tissues), glutathione peroxidase 3 (in plasma mammalian tissues), lactoperoxidase, myeloperoxidase (in salivary & mucosal mammalian tissues), myeloperoxidase (in neutrophil mammalian tissues), cytochrome peroxidase (in yeasts such as Candida albicans) and horseradish peroxidase (common to show in vitro activity). One of skill will appreciate that oxidoreductases are selective and, in some embodiments, the oxidoreductase can include an alternate enzyme that are selective for a binding system having a phenolic compound that acts as a substrate for the alternative enzyme.

In some embodiments, the oxidoreductases include mono-oxygenases such as, for example, phenylalanine monooxygenase, tyrosine monooxygenase, and tryptophan monooxygenase. In some embodiments, the oxidoreductases include dioxygenases such as, for example, tryptophan dioxygenase, homogentisate dioxygenase, trimethyl lysine dioxygenase, and nitric oxide synthase. In some embodiments, the oxidoreductases include peroxidases such as, for example, catalase, myeloperoxidase, thyroperoxidase. In some embodiments, the oxidoreductases act in the presence of a co-factor or co-enzyme, such as nicotinamide adenine dinucleotide phosphate (NADP) or nicotinamide adenine dinucleotide (NAD).

### Methods of Making the Compositions

The design of the formulations includes (i) selecting the agent, (ii) selecting the reactive oxygen species, (iii) selecting the ratio of agent to reactive oxygen species, and (iv) selecting a carrier. In some embodiments, the agent can be derivatized or attached to another chemical moiety via a linker, or another known method such as, for example, esterification to facilitate or improve an association between the agent and the reactive oxygen species, as well as to potentially modify, solubility, tissue absorption, or toxicity. And, in some embodiments, the agent can include a combination of phenolic compound species. For example, a first agent can be in combination with a second agent in a combination ranging from about 1:1000 to about 1000:1, from about 1:1000 to about 100:1, from about 1:1000 to about 10:1, from about 1:1000 to about 1:1, from about 1:10 to about 10:1, from about 1:9 about 9:1, from about 1:8 about 8:1, from about 1:7 about 7:1, from about 1:6 about 6:1, from about 1:5 about 5:1, from about 1:4 about 4:1, from about 1:3 about 3:1, from about 1:2 about 2:1, from about 1:1.5 about 1.5:1, or any range therein.

One of skill will appreciate that, at least from the teachings provided herein, there are a vast number of components that can be selected, the selection of which is, at least in part, dependent on type of enzyme, co-enzymes, cofactors or catalysts present at the target site for the bioactivation of the system. The design of the system can include for example, (i) identifying the target site; (ii) identifying an enzyme, co-enzymes, cofactors, or catalysts present at the target site but not present at tissue surrounding the target site; (iii) selecting a binding pair for activation at the target site by the enzyme, co-enzymes, cofactors, or catalysts; and, (iv) selecting a carrier in which the binding pair is stable or substantially stable. Identifying the target site can include, for example, select a target tissue for treatment, such as a spastic tissue at which the enzyme, co-enzymes, cofactors or catalysts present. In some embodiments, the target site is a GI tissue, at which peroxidase or oxidase may be present. Identifying an enzyme, co-enzymes, cofactors, or catalysts present at the target site but not present at tissue surrounding the target site can include, for example, identifying the tissue type, as well as the presence of a microbe. Anaerobic pathogens such as Pseudomonas and Vibrio, for example, can express a peroxide or an oxidase, making these enzymes available at the target site.

After the system and environment of use are known, one of skill can select a carrier in which the formulation is stable or substantially stable. In one example, the formulation can comprise a mixture of one or more phenolic compounds in a desired ratio with hydrogen peroxide. For example, the phenolic compounds can include a mixture of a plant extract, such as a pomegranate extract and/or a green tea extract, and the ratio of agent to hydrogen peroxide can range from about 1:2 to about 1:20 on a wt/wt basis, which can include molar weight bases. In some embodiments, the hydrogen peroxide can be added to the agent using a concentration of about 0.01% to about 10% hydrogen peroxide solution, and any free hydrogen peroxide can remain or be removed using the teachings provided herein. One of skill can easily select the dose for a particular use, which will vary according to factors that include the environmental conditions at the site of use. In another example, the formulations can comprise a mixture of agents in a desired ratio with hydrogen peroxide. For example, the agents can include a mixture of a pomegranate extract and a green tea extract, and the ratio of phenolic compound to hydrogen peroxide can range from about 3:1 to about 1:3 on a wt/wt basis (e.g., molar weight). The hydrogen peroxide can be added to the agent using a concentration of about 0.01% to about 10% hydrogen peroxide. In some embodiments, a 35% hydrogen peroxide stock solution can be used as a source of hydrogen peroxide, which can be obtained from a commercially available stock solution, for example. In some embodiments, up to 60% hydrogen peroxide stock solution can be used as a source of hydrogen peroxide. In fact, higher concentrations are available, and could be used in some embodiments if handled properly. One of skill will be able to readily select, obtain and/or produce desired concentrations of hydrogen peroxide. Again, one of skill can easily select the dose for a particular use, which will vary according to factors that include environmental conditions at the site of use. In some embodiments, this formulation has worked well for uses in animals that are non-humans.

In some embodiments, the phenolic compound can be a polyphenolic, or a mixture of polyphenolics. The compositions can include, for example, a weight (molar or mass) ratio of phenolic compound to reactive oxygen species that ranges from about 1:1000 to about 1000:1. In some embodiments, the ratio of phenolic compound to reactive oxygen species can range from about 1:1000 to about 500:1, from about 1:500 to about 500:1, from about 1:250 to about 500:1, from about 1:500 to about 250:1, from about 1:250 to about 250:1, from about 1:100 to about 250:1, from about 1:250 to about 100:1, from about 1:100 to about 100:1, from about 1:100 to about 50:1, from about 1:50 to about 100:1, from about 1:50 to about 50:1, from about 1:25 to about 50:1, from about 1:50 to about 25:1, from about 1:25 to about 25:1, from about 1:10 to about 10:1, from about 1:1000 to about 250:1, from about 1:1000 to about 100:1, from about 1:1000 to about 50:1, from about 1:1000 to about 25:1, from about 1:1000 to about 10:1, from about 1:1000 to about 5:1, from about 1:10 to about 1:20, from about 1:10 to about 1:30, from about 1:10 to about 1:40, from about 1:10 to about 1:50, from about 1:10 to about 1:60, from about 1:10 to about 1:70, from about 1:10 to about 1:80, from about 1:10 to about 1:90, from about 1:20 to about 1:30, from about 1:20 to about 1:40, from about 1:20 to about 1:50, from about 1:20 to about 1:60, from about 1:20 to about 1:70, from about 1:20 to about 1:80, from about 1:20 to about 1:90, from about 1:30 to about 1:90, or any range therein. In some embodiments, the reactive oxygen species can include hydrogen peroxide, alone or in combination with other reactive oxygen species.

In some embodiments, the formulation comprises a ratio of a tannin and hydrogen peroxide, a phenylpropanoid and a hydrogen peroxide, a catechin and hydrogen peroxide, an epigallic acid and a hydrogen peroxide, or a combination thereof an of these phenolic compounds with hydrogen peroxide.

In some embodiments, the compositions include a stable hydrogen bonded complex between the phenolic compound and the reactive oxygen species. For example, a highly hydroxylated polyphenol compound can be combined with a high concentration of hydrogen peroxide, the combination leading to binding the hydrogen peroxide to the phenolic compound to produce the binding system. The binding system can be intended for dilution in water or a solid excipient. One of skill will appreciate that such a complex can be referred to as a polyphenol peroxysolvate, in some embodiments, when in a liquid form for storage or administration to a subject, and a phenolic perhydrate when in an anhydrous, or substantially anhydrous, form for storage or administration to a subject.

The formulations can be carried as a liquid, powder, capsule, tablet, or gas for administration to a subject. The selection of the phenolic compound should take into consideration the manner in which the reactive oxygen species will bind to the agent to form a stable, or substantially stable combination. The combination can be considered substantially stable where the reactive oxygen species retains all, most, or at least a predictable amount of oxidation strength for the uses and functions recited herein.

One of skill will appreciate that an agent, such as a phenolic or polyphenolic compound, can be derivatized to introduce or enhance a desired function. The agent can be derivatized, for example, to increase it's functionality for binding to the reactive oxygen species, maintaining stability or miscibility in a carrier, or binding to a target site, using any method known to one of skill. In some embodiments, the agent can be bound to a polyol, pegylated, attached to a saccharide, or attached to glucose, for example.

Moreover, one of skill will appreciate that the formulations should, in some embodiments, be produced free of compounds that can lead to degradation of the otherwise stable, or substantially stable, combinations. As such, in some embodiments, the formulations comprise solutes that are substantially free of transition metals, metal ions, heavy metals, oxidoreductase enzymes, other strong oxidizers, reactive halogen compounds, hydrogen halides, and other compounds that can cause a decomposition of the reactive oxygen species, or its disassociation from the agent with which it forms a combination.

The formulations can be made using ingredients from commercially available chemical providers, such as individual chemical compounds, mixtures of chemical compounds, or plant extracts; or, they can be made directly as an extract of a plant tissue, for example, a water extract, an alcohol extract, or a combination thereof. In some embodiments, the ingredients can be a nano-pulverized powder of a chemical compound, mixture of compounds, a plant extract, or a combination thereof. In some embodiments, for example, the agent can include a chemical compounds that is commercially available. In some embodiments, the chemical compounds are synthetically produced, recombinantly produced, and/or derivatized. In some embodiments, a plant extract can be combined with such a chemical compound as an additional agent at a desired ratio to enhance performance or design a particular desired therapeutic activity or combination of therapeutic activities.

Commercially Available Sources

Commercially available chemical providers, for example Sigma-Aldrich, can provide agents, such as phenolic and polyphenolic chemicals, for use with the methods and formulations taught herein. In the example set forth below, (i) gallic acid (a model polyphenol building block) is combined with hydrogen peroxide; and, (ii) tannic acid (a model polyphenol component) is combined with hydrogen peroxide. Both gallic acid and tannic acid are commercially available from Sigma-Aldrich. One of skill will appreciate that a wide variety of polyphenolics are commercially available.

A Whole, Plant Extract as a Source of the Phenolic Component

The method of obtaining the phenolic component, e.g, the polyphenol component, from a plant tissue can be produced using a combination of the following steps:
i. Harvest plant tissue comprising a polyphenol component, for example, the polyphenol comprising a tannin. It is desirable to harvest while minimizing physical damage to the plant tissue. For example, whole leaf extractions can be performed to avoid physical damage to the leaves, but it may be desirable to reduce the size of the leaves by cutting them, for example, to increase the speed and yield of the extraction in some embodiments.
ii. Denaturing all, or substantially all, of the oxidoreductase enzymes in the plant. This can be done through drying, for example, using heating in the range of about 60° C. to about 150° C., or a combination of such heating and dessication. Alcohols can also be used to denature the enzymes.
iii. Extracting the polyphenols from the plant tissue using a suitable solvent including, but not limited to, water or an alcohol. Water extractions have been used in this example. Since we're after water soluble plant materials, a simple water extraction is sufficient to provide the plant extract containing polyphenols for the compositions.

The plant extraction procedures are simple, although they can be modified for efficiency in product yield and activity. Although inefficient, a simple extraction procedure, for example, would be to merely harvest the plant tissue and soak the tissue in water to isolate the water soluble extract of the plant tissue In some embodiments, one might harvest the plant tissue, denature the endogeneous enzymes to at least substantially inactivate the enzymes, and soak the tissue in water to isolate the water soluble extract of the plant tissue. It was observed that the therapeutic activity of the binding systems increased in a surprising and unexpected amount after at least substantially inactivating the endogenous enzymes. Another simple extraction method would be to harvest the plant tissue, and isolate the water soluble extract of the tissue in water at temperatures greater than about 80° C. to steam. As such, simpler processes may not include denaturing the enzymes, but the stability and activity of the extract in the composition can be expected to suffer greatly in some embodiments. Additional steps can be added, however, to increase the efficiency of the extraction, although such steps are not required. For example, the harvesting can include cutting into as large of pieces as practical to the size of the plant to preserve the metabolic activity in the plant tissue can be done. The plant tissue can be pulverized after denaturing the enzymes, and the water can be heated at temperatures ranging from about 25° C. to about 100° C., from about 30° C. to about 95° C., from about 35° C. to about 90° C., from about 40° C. to about 85° C., from about 45° C. to about 80° C., from about 45° C. to about 75° C., from about 45° C. to about 70° C., from about 45° C. to about 65° C., or any amount or range therein in increments of 1° C., to make the process of extraction more efficient.

In some embodiments, the endogeneous enzymes include a catalase or peroxidase that is at least substantially inactivated. In some embodiments, the endogeneous enzymes can be inactivated through heating, cooling, boiling, freezing, dessicating, freezing and thawing cycles, blanching, or a combination thereof. In some embodiments, the endogeneous enzymes can be inactivated using a process that includes allowing natural degradation over time, adding at least 1% salt, radiating, or adding an exogeneous chemical enzymatic inhibitor.

In some embodiments, the plant extract is produced from a process comprising: harvesting the plant tissue; at least partially inactivating an endogeneous enzyme; optionally reducing particle size of the plant tissue through cutting, avulsing, or pulverizing; creating the extracted component through a process that includes combining the plant tissue with water or alcohol for an effective time and at an effective temperature; optionally removing particles from the mixture; and, adding the reactive oxygen species to the effective, or otherwise desired, amount.

In some embodiments, the water soluble plant extract can then be optionally filtered, for example, using a filter, for example, a 5 um filter in some embodiments, and hydrogen peroxide can then be added to the filtered extract to a concentration of 1% by weight of the total composition. In some embodiments, the filter used can be a 0.1 um, 0.5 um, 1 um, 2 um, 3 um, 4 um, 5 um, 6 um, 7 um, 8 um, 9 um, 10 um, 11 um, 12 um, 13 um, 14 um, 15 um, 20 um, or any size therein in increments of 0.1 um, filter.

In some embodiments, the hydrogen peroxide can be added to the extract in an amount ranging from about 0.01% by weight to about 10% by weight of the total composition. As such, the amount of hydrogen peroxide added to the agent can be about 0.01%, about 0.02%, about 0.03%, about 0.04%, about 0.05%, about 0.10%, about 0.20%, about 0.30%, about 0.40%, about 0.50%, about 1.0%, about 1.2%, about 1.4%, about 1.6%, about 1.8%, about 2.0%, about 3.0%, about 4.0%, about 5.0%, about 6.0%, about 7.0%, about 8.0%, about 9.0%, about 10.0%, or any amount therein in increments of 0.01%. Increasing the concentration of hydrogen peroxide added has been observed to increase the potency and stability of the resulting compositions.

After combining the reactive oxygen species with the phenol component, such as one or more polyphenols, the free reactive oxygen species in the compositions can be left in the composition, or it can be removed using an enzyme, catalyst, or reducing agent. In this example, the reactive oxygen species is hydrogen peroxide, and the free hydrogen peroxide can be removed from the composition in a subsequent step contacting the free hydrogen peroxide with a hydrogen peroxide degrading enzyme, such catalase; a catalyst such as manganese dioxide, platinum, iron, or copper; or, a reducing agent such as ferric chloride, copper sulfate, or sodium hypochlorite. In some embodiments, the composition having the free hydrogen peroxide can be contacted with a metal catalyst or catalase bound to a solid non-soluble substrate. In some embodiments, the solid substrate can be a bead column or screen, for example. Likewise, the catalysts and reducing agents can be used in a similar manner to remove the free hydrogen peroxide, or any other free reactive oxygen species.

As such, the concentration of free reactive oxygen species, such as free hydrogen peroxide, remaining in the composition can range from about 0 to about 10% based on total dry weight of the composition. Moreover, in some embodiments, the total hydrogen peroxide concentration can range from about 0.001% to about 1%, from about 0.001% to about 0.1%, from about 0.01% to about 0.05%, from about 0.005% to about 5%, from about 0.007% to about 2%, from about 0.01% to about 5%, from about 0.05% to about 5%, from about 0.1% to about 5%, from about 0.2% to about 4.5%, from about 0.3% to about 4%, from about 0.4% to about 3.5%, from about 0.5% to about 3%, from about 0.6% to about 2.5%, from about 0.7% to about 2%, from about 0.001% to about 1.5%, about 1%, or any amount or range therein in increments of 0.001%. And, it should be appreciated that the concentration of free hydrogen peroxide, for example, can also be reduced, or further reduced, by dilution of the composition in various commercial formulations.

Moreover, precipitates of protein or other impurities can form at this point and can optionally be removed by additional filtration, and we often filter after we allow the solution to react for about an hour. Although not necessary, additional reactive oxygen species can be added to ensure complete saturation of hydrogen peroxide on the binding sites of the polyphenols in the extract. In this example, hydrogen peroxide was used as the reactive oxygen species, keeping track of the total hydrogen peroxide concentration.

The plant extract can be combined with the reactive oxygen species to form a suspension in some embodiments, or a solution in some embodiments. It should be appreciated that, in some embodiments, only a solution is used. The suspension or solution can be allowed to react for a period of time ranging from about 10 minutes to about 72 hours, in some embodiments, before diluting the composition to a desired concentration. In some embodiments, the solution can be allowed to react for a period of time ranging from about 1 minute to about 96 hours, from about 5 minutes to about 48 hours, from about 10 minutes to about 36 hours, from about 10 minutes to about 24 hours, from about 10 minutes to about 12 hours, from about 10 minutes to about 8 hours, or from about 10 minutes to about 1 hour, or any range therein in increments of 1 minute. In this example, the extracts were allowed to react with the hydrogen peroxide for a minimum of 2 hours. The dilution can be desirable, for example, (i) to control the concentration of the composition in solution, and/or (ii) to accelerate degradation of the unbound reactive oxygen species to limit the composition to having no, or substantially no, free reactive oxygen species. In this example, the hydrogen peroxide is more susceptible to degradation when free in solution, and one of skill will appreciate that the degradation will increase in rate when the composition is diluted.

In some embodiments, dry compositions are provided. For example, the system can be in the form of a powder, pill, tablet, capsule, or as separate dry components for mixing into a liquid form. In these embodiments, for example, both a phenolic compound and a reactive oxygen species can be in a dry form either before or after creation of the binding pair, and the binding system can be used in the dry form, or converted to a liquid form, for any of the uses taught herein. The advantages of the dry compositions can include, for example, the ease of storage and transport. In some embodiments, the binding systems, whether in liquid or dry form, can be combined with vitamins, electrolytes, and/or other nutrients in either liquid or dry form. The dry form of the binding system can be manufactured using any drying process known to one of skill, such as solvent exchange, vacuum drying, critical point drying, heating, dessication, or a combination thereof. In some embodiments, the phenolic compound is dried as a single component. In some embodiments, the binding pair is formed, and the binding pair is dried together. And, in some embodiments, the reactive oxygen species can be, independently, in any dry form known to one of skill, such as the dry forms taught herein. In embodiments having the reactive oxygen species in an independent dry form, the dry phenolic compound and the dry reactive oxygen species can be combined in a polar solvent, for example, to create the binding pair prior to use.

### Methods of Using the Compositions

In some embodiments, the binding systems can be administered for inhibiting the growth of, or killing, antibiotic-resistant bacteria such as, for example, spore-forming, anaerobic antibiotic-resistant bacteria. In some embodiments, the antibiotic-resistant bacteria are endospores. Examples of endospores can include Bacillus and Clostridium. In some embodiments, the antibiotic-resistant bacteria include endospores that can be any one, or any combination of, Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and Vulcanobacillus,

In particular, one of skill will appreciate having compositions and methods of killing Clostridium difficile (C. diff). One of skill would appreciate a reliable method of treating C. diff-induced conditions such as, for example, diarrhea and intestinal inflammation, without eradicating normal gut flora or promoting of antibiotic resistance. For at least the reasons discussed above, one of skill will appreciate the teachings provided herein, which include (i) methods of avoiding or reducing the use of antibiotics; (ii) direct mechanisms of reducing C. diff virulence; and (iii) indirect mechanisms of increasing host immunity. Such compositions and methods help, for example, to meet a growing need for effective control of hospital acquired infections (HAIs) resulting from antibiotic-resistant pathogens generally associated with the selective pressure induced by the frequent use of antibiotics. It will be appreciated that the compositions and methods taught herein are an alternative to the use of antibiotics, representing a paradigm shift that reduces clinical symptoms of HAIs without invoking the problematic antibiotic resistance mechanisms that have become such a serious problem to our society.

This can include reducing or eliminating abdominal pain, bloating, forceful defecation, forceful vomiting, defecation urgency, constipation, and/or incontinence. Such symptoms can arise from mild conditions to serious conditions such as, for example, food poisoning, constipation, gastroenteritis, viral infections, bacterial infections, lactose intolerance, excessive flatulence and bloating, indigestion, diverticulitis, autoimmune disease, intestinal inflammation and even colorectal cancer, adhesions, and the like.

The compositions taught herein can be used in treating such conditions, either alone or in co-administrations with nutritional therapy or rehydration therapies. In some embodiments, the composition can be co-administered with at least one other nutritional and/or rehydrating agent for aiding recovery from a health imbalance, or to maintain a health balance. Examples of rehydrating agents can include, but are not limited to, GATORADE and other electrolyte drinks, oral rehydration solutions (ORSs) generally, new oral rehydration solution (N-ORS), SEURO ORAL, PEDIAONE, and PEDIALYTE. Examples of nutritional supplements can include, but are not limited to, zinc sulfate, salted rice water, salted yogurt-based drinks, and vegetable or chicken soup with salt. Such health imbalances can include, but is not limited to, dehydration, malnutrition, electrolyte imbalance, vitamin deficiency, food hypersensitivities, stress-induced diarrhea, abdominal cramping, or a combination thereof. In some embodiments, the methods taught herein can further include the administration of oral rehydrating or nutritional agents such as sodium, potassium, dextrose, fructose, glucose, magnesium, zinc, selenium, vitamin A, Vitamin D, Vitamin C, dietary fiber, and combinations thereof. The amounts and ratios of the agents to the composition can be substantially varied to provide prophylaxis, therapy or maintenance of healthful balance. Ratios of the compositions herein to the nutritional agents or rehydration agents can range, for example, from about 1:100 to about 100:1, from about 1:50 to about 50:1, from about 1:40 to about 40:1, from about 1:30 to about 30:1, from about 1:20 to about 20:1, from about 1:10 to about 10:1, from about 1:5 to about 5:1, from about 1:4 to about 4:1, from about 1:3 to about 3:1, from about 1:2 to about 2:1, from about 1:1.5 to about 1.5:1, about 1:1, or any range therein. The ratios can be based on volume:volume, mass:volume, volume:mass, mass:mass, or molar:molar. It should be appreciated that the concentrations of the compositions taught herein can be the same or different than the concentrations of the nutritional agents or rehydration agents. And, it should also be appreciated that the concentrations and ratios of concentrations can be subjective to a particular administration, such that they can be independently selected according to the condition treated, objective sought, desired effect, and/or personal preference. The combinations can be administered under any regime taught herein for the administration of an agent or combination of agents.

The targeted action of the binding systems allows for the administration of surprisingly low effective doses of the phenolic compounds. As a result, the compositions also improve safety by substantially increasing the separation between an effective dose and any toxic/side effects.

The terms “treat,” “treating,” and “treatment” can be used interchangeably and refer to the administering or application of the binding systems taught herein, including such administration as a health or nutritional supplement, and all administrations directed to the prevention, inhibition, amelioration of the symptoms, or cure of a condition taught herein. The terms “disease,” “condition,” “disorder,” and “ailment” can be used interchangeably in some embodiments. The term “subject” and “patient” can be used interchangeably and refer to an animal such as a mammal including, but not limited to, non-primates such as, for example, a cow, pig, horse, cat, dog, rat and mouse; and primates such as, for example, a monkey or a human. As such, the terms “subject” and “patient” can also be applied to non-human biologic applications including, but not limited to, veterinary, companion animals, commercial livestock, aquaculture, and the like.

In some embodiments, the composition includes (i) a phenolic compound selected from the group consisting of condensed tannins, hydrolysable tannins, complex tannins, phlorotannins, psuedotannins, and derivatives thereof; and, (ii) hydrogen peroxide in a stable, or substantially stable, non-covalent association.

In some embodiments, the compositions taught herein can be used to protect, maintain, improve, or restore a digestive health of a subject when administered orally in an effective amount. In some embodiments, the effectiveness can be measured by comparing to a control group that did not receive the administration of the compositions taught herein. And, in some embodiments, the effectiveness can be measured according to a historical baseline for the subject being treated.

As such, the compositions taught herein can be used to prevent or inhibit the loss of digestive tract homeostasis, or ameliorate the symptoms associated with a loss of digestive tract homeostasis. In some embodiments, the binding systems can be used to prevent, treat, ameliorate the symptoms of, or even cure, a chronic gastrointestinal condition.

Such conditions can include, but are not limited to, hyperacidity, colitis, irritable bowel syndrome, Crohn's disease, necrotic enteritis, functional colonic diseases, malabsorption, a peptic ulcer, gastro-esophageal reflux disease, ulcerative colitis, and diverticulitis. In some embodiments, the binding systems can be used to reduce mucosal tissue inflammation, dysfunction, or damage. Such conditions can be induced, for example, by drug side-effects, chemotherapy, dysbiosis, radiation, changes in normal flora, hyperimmunity, autoimmune reactions, immune deficiencies, nervousness, allergies, chemical irritation, and stress.

In some embodiments, the binding systems can be administered for selectively inhibiting the growth of gastrointestinal pathogens. It should be appreciated that there may be lesser inhibition of non-pathogenic strains, particularly common probiotic bacteria such as bifidobacteria and lactobacilli. And, in some embodiments, administration of the binding systems can produce short term immune modulation effects as well as potentially change the chronic expression of the activating enzymes associated with some conditions with longer term use of the binding systems.

In some embodiments, the symptoms of a gastrointestinal condition can include, for example, diarrhea, dehydration, malnutrition, constipation, nausea, and/or cramping. And, in some embodiments, the symptoms of a gastrointestinal condition can be temporary and include acid irritation, indigestion, bloating, cramps, spasmodic peristalsis, diarrhea, and constipation. Administering the compositions and formulations taught herein for the treatment and/or management of gastrointestinal conditions can be considered a nutritional or health supplement, in some embodiments. In some such embodiments, for example, the compositions and formulations taught herein can be administered to prevent, inhibit, or ameliorate the effect, infectivity, and virulence of pathogens including bacteria, virus, fungi, yeast, prions, protozoa and parasites in a subject orally taking an effective amount of the supplement.

As described herein, the compositions and formulations taught herein can be used in a method of treating acute diarrhea in a subject. In some embodiments, the methods comprise orally administering an effective amount of a binding system taught herein to the subject. The compositions and formulations taught herein can prevent, inhibit, or ameliorate a symptom of acute diarrhea in the subject when compared to a second subject in a control group in which the binding system was not administered. In some embodiments, the symptom is selected from the group consisting of a stool score, heartburn, indigestion, urgency of defecation, nausea, vomiting, stomach pain, and bloating.

As described herein, the compositions and formulations taught herein can be used in a method of treating food poisoning in a subject. In some embodiments, the method comprises orally administering an effective amount of a composition or formulation taught herein taught herein to the subject. The binding system can prevent, inhibit, or ameliorate the symptoms of food poisoning in the subject when compared to a second subject in a control group in which the binding system was not administered. In some embodiments, the symptom is selected from the group consisting of a stool score, heartburn, indigestion, urgency of defecation, nausea, vomiting, stomach pain, and bloating.

### Methods of Administering the Compositions

The terms “administration” or “administering” can be used to refer to a method of incorporating a composition into the cells or tissues of a subject, either in vivo or ex vivo to test the activity of a system, as well as to diagnose, prevent, treat, or ameliorate a symptom of a disease. In one example, a compound can be administered to a subject in vivo using any means of administration taught herein. In another example, a compound can be administered ex vivo by combining the compound with cell tissue from the subject for purposes that include, but are not limited to, assays for determining utility and efficacy of a composition. And, of course, the systems can be used in vitro to test their stability, activity, toxicity, efficacy, and the like. When the compound is incorporated in the subject in combination with one or active agents, the terms “administration” or “administering” can include sequential or concurrent incorporation of the compound with the other agents such as, for example, any agent described above. A pharmaceutical composition of the invention can be formulated, in some embodiments, to be compatible with its intended route of administration.

Any administration vehicle known to one of skill to be suitable for administration of the compounds, compositions, and formulations taught herein can be used. A “vehicle” can refer to, for example, a diluent, excipient or carrier with which a compound is administered to a subject. A “pharmaceutically acceptable carrier” is a diluent, adjuvant, excipient, or vehicle with which the composition is administered. A carrier is pharmaceutically acceptable after approval by a state or federal regulatory agency or listing in the U.S. Pharmacopeial Convention or other generally recognized sources for use in subjects. The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

The compositions can be administered to a subject orally or rectally, for example, in the maintaining or restoring of digestive homeostasis. Oral administration can include digestive tract, buccal, sublingual, and sublabial, and a carrier such as a solid or liquid can be used. A solid can include, for example, a pill, capsule, tablet, or time-release technology in some embodiments; and, for buccal or sublingual, a solid can include an orally disintegrating tablet, a film, a lollipop, a lozenge, or chewing gum; and, a liquid can include a mouthwash, a toothpaste, an ointment, or an oral spray. A liquid can include, for example, a solution, soft gel, suspension, emulsion, syrup, elixir, tincture, or a hydrogel.

Tablets, pills, capsules, troches liquids and the like may also contain binders, excipients, disintegrating agent, lubricants, glidants, chelating agents, buffers, tonicity modifiers, surfactants, sweetening agents, and flavoring agents. Some examples of binders include microcrystalline cellulose, gum tragacanth or gelatin. Some examples of excipients include starch or maltodextrin. Some examples of disintegrating agents include alginic acid, corn starch and the like. Some examples of lubricants include magnesium stearate or potassium stearate. An example of a chelating agent is EDTA. Some examples of buffers are acetates, citrates or phosphates. Some examples of tonicity modifiers include sodium chloride and dextrose. Some examples of surfactants for micellation or increasing cell permeation include coconut soap, anionic, cationic or ethoxylate detergents. An example of a glidant is colloidal silicon dioxide. Some examples of sweetening agents include sucrose, saccharin and the like. Some examples of flavoring agents include peppermint, chamomile, orange flavoring and the like. It should be appreciated that the materials used in preparing these various compositions should be pharmaceutically pure and non-toxic in the amounts used

Rectal administrations can be made using any method known to one of skill. For example, a suppository formulation can be prepared by heating glycerin to about 120° C., combining the binding system with the heated glycerin, mixing the combination, adding purified water to a desired consistency, and pouring the desired consistency into a mold to form the suppository.

The compositions may be administered as suspensions or emulsions. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions. In some embodiments, an administration, such as an oral or rectal administration, for example, may include liposomes. In some embodiments, the liposome may assist in a targeted delivery system. The liposomes can be designed, for example, to bind to a target protein and be taken up selectively by the cell expressing the target protein.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer, and the like. Such formulations can generally be prepared using methods known to one of skill in the art.

In some embodiments, the compositions and formulations taught herein can be administered in a sustained release formulation, and the formulation can include one or more agents in addition to the composition. In some embodiments, the sustained release formulations can reduce the dosage and/or frequency of the administrations of such agents to a subject. In some embodiments, an exogenous catalyst or enzyme is introduced to a target and one or more of the reactive oxygen species, phenolic compound, or the exogeneous catalyst or enzyme are segregated by encapsulation or micellation to delay the bioactivation until target site is reached by all components.

One of skill understands that the amount of the agents administered can vary according to factors such as, for example, the type of disease, age, sex, and weight of the subject, as well as the method of administration. For example, an administration can call for substantially different amounts to be effective. Dosage regimens may also be adjusted to optimize a therapeutic response. In some embodiments, a single bolus may be administered; several divided doses may be administered over time; the dose may be proportionally reduced or increased; or, any combination thereof, as indicated by the exigencies of the therapeutic situation and factors known one of skill in the art. It is to be noted that dosage values may vary with the severity of the condition to be alleviated, as well as whether the administration is prophylactic, such that the condition has not actually onset or produced symptoms. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners. The compounds can be administered in dosage units. The term “dosage unit” can refer to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

An “effective amount” of a compound can be used to describe a therapeutically effective amount or a prophylactically effective amount. An effective amount can also be an amount that ameliorates the symptoms of a disease. A “therapeutically effective amount” can refer to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount should be administered in an amount sufficient to result in amelioration of one or more symptoms of a disorder, prevention of the advancement of a disorder, or regression of a disorder. In some embodiments, for example, a therapeutically effective amount can refer to the amount of an agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. In some embodiments, the effectiveness can be measured by comparing to a control group that did not receive the administration of the compositions taught herein. And, in some embodiments, the effectiveness can be measured according to a historical baseline for the subject being treated.

In some embodiments, the desired action of the composition is relief of a gastrointestinal spasm. In some embodiments, the desired action can include, for example, reducing or eliminating abdominal pain, bloating, forceful defecation, forceful vomiting, defecation urgency, constipation, and/or incontinence. In these embodiments, at least 10% relief can be obtained in a time ranging from 1 minute to 24 hours, from about 5 minutes to about 18 hours, from about 10 minutes to about 12 hours, from about 20 minutes to about 8 hours, from about 30 minutes to about 6 hours, from about 1 hours to about 4 hours, from about 2 hours to about 10 hours, from about 3 hours to about 9 hours, or any range or amount therein in increments of 5 minutes.

A “prophylactically effective amount” can refer to an amount that is effective at the dosages and periods of time necessary to achieve a desired prophylactic result, such as prevent the onset of an inflammation, allergy, nausea, diarrhea, infection, and the like. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

In some embodiments, a therapeutically or prophylactically effective amount of a composition may range in concentration from about 0.01 nM to about 0.10 M; from about 0.01 nM to about 0.5 M; from about 0.1 nM to about 150 nM; from about 0.1 nM to about 500 μM; from about 0.1 nM to about 1000 nM, 0.001 μM to about 0.10 M; from about 0.001 μM to about 0.5 M; from about 0.01 μM to about 150 μM; from about 0.01 μM to about 500 μM; from about 0.01 μM to about 1000 nM, or any range therein. In some embodiments, the compositions may be administered in an amount ranging from about 0.005 mg/kg to about 100 mg/kg; from about 0.005 mg/kg to about 400 mg/kg; from about 0.01 mg/kg to about 300 mg/kg; from about 0.01 mg/kg to about 250 mg/kg; from about 0.1 mg/kg to about 200 mg/kg; from about 0.2 mg/kg to about 150 mg/kg; from about 0.4 mg/kg to about 120 mg/kg; from about 0.15 mg/kg to about 100 mg/kg, from about 0.15 mg/kg to about 50 mg/kg, from about 0.5 mg/kg to about 10 mg/kg, or any range therein, wherein a human subject is often assumed to average about 70 kg.

In some embodiments, the concentration of the agent ranged in dry weight from 1 μg/ml to 5000 μg/ml, or any range therein. In some embodiments, the concentration in dry weight was about 1 μg/ml, about 5 μg/ml, about 10 μg/ml, about 15 μg/ml, about 20 μg/ml, about 25 μg/ml, about 30 μg/ml, about 35 μg/ml, about 40 μg/ml, about 45 μg/ml, about 50 μg/ml, about 60 μg/ml, about 70 μg/ml, about 80 μg/ml, about 90 μg/ml, about 100 μg/ml, about 125 μg/ml, about 150 μg/ml, about 175 μg/ml, about 200 μg/ml, about 250 μg/ml, about 300 μg/ml, about 350 μg/ml, about 400 μg/ml, about 450 μg/ml, about 500 μg/ml, about 550 μg/ml, about 600 μg/ml, about 650 μg/ml, about 700 μg/ml, about 750 μg/ml, about 800 μg/ml, about 850 μg/ml, about 900 μg/ml, about 950 μg/ml, about 1000 μg/ml, about 1250 μg/ml, about 1500 μg/ml, about 1750 μg/ml, about 2000 μg/ml, about 2500 μg/ml, about 3000 μg/ml, about 3500 μg/ml, about 4000 μg/ml, about 4500 μg/ml, about 5000 μg/ml, or any concentration therein in increments of 1 μg/ml,

The amount of the composition administered may vary widely depending on the type of formulation, size of a unit dosage, kind of excipients, and other factors well known to those of ordinary skill in the art. A formulation may comprise, for example, an amount of the composition ranging from about 0.0001% to about 6% (w/w), from about 0.01% to about 1%, from about 0.1% to about 0.8%, or any range therein, with the remainder comprising the excipient or excipients. In some embodiments, the compositions can be administered, for example, in an amount of ranging from about 0.1 μg/kg to about 1 mg/kg, from about 0.5 μg/kg to about 500 μg/kg, from about 1 μg/kg to about 250 μg/kg, from about 1 μg/kg to about 100 μg/kg from about 1 μg/kg to about 50 μg/kg, or any range therein. One of skill can readily select the frequency and duration of each administration. For example, depending on the gastrointestinal disorder treated, whether a prophylactic treatment or a treatment of an existing disorder, variables such as the age and size of the subject can be considered, as well as the source and type of the polyphenol component and the intensity of the symptoms. In some embodiments, the compositions can be administered orally in daily doses ranging from about 5 μg to about 5000 μg dry weight, for example. In such embodiments, the compositions can be administered orally in amounts ranging from about 5 μg to about 5000 μg, from about 10 μg to about 4000 μg, from about 20 μg to about 3000 μg, from about 50 μg to about 2000 μg, from about 100 μg to about 1000 μg, from about 250 μg to about 500 μg, or any range therein, in dry weight. In some embodiments, the compositions can be administered orally in daily doses of about 100 μg, about 200 μg, about 300 μg, about 400 μg, about 500 μg, about 600 μg, about 700 μg, about 800 μg, about 900 μg, about 1000 μg, about 2000 μg, about 3000 μg, about 4000 μg, about 5000 μg, about 6000 μg, about 7000 μg, about 8000 μg, about 9000 μg, or any range or amount therein in increments of 1.0 μg dry weight.

In some embodiments, the compositions can be administered in daily doses ranging from about 0.1 μg/kg to about 500 μg/kg dry weight, for example. For example, in some embodiments, the compositions can be administered orally in amounts ranging from about 0.1 μg/kg to about 500 μg/kg, from about 0.2 μg/kg to about 200 μg/kg, from about 0.3 μg/kg to about 300 μg/kg, from about 0.4 μg/kg to about 400 μg/kg, from about 0.5 μg/kg to about 500 μg/kg, from about 1.0 μg/kg to about 100 μg/kg, from about 2 μg/kg to about 100 μg/kg, from about 3 μg/kg to about 100 μg/kg, from about 4 μg/kg to about 100 μg/kg, from about 5 μg/kg to about 100 μg/kg, from about 6 μg/kg to about 100 μg/kg, from about 7 μg/kg to about 100 μg/kg, from about 8 μg/kg to about 100 μg/kg, from about 9 μg/kg to about 100 μg/kg, from about 10 μg/kg to about 100 μg/kg, from about 1.0 μg/kg to about 50 μg/kg, from about 1.0 μg/kg to about 25 μg/kg, from about 1.0 μg/kg to about 10 μg/kg, or any range or amount therein in increments of 1.0 μg/kg dry weight. In some embodiments, the compositions can be administered in daily doses of about 1 μg/kg, about 2 μg/kg, about 3 μg/kg, about 4 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, or any range therein in increments of 1.0 μg/kg.

It should be appreciated that the doses can be administered once a day, twice a day, three times a day, four times a day, five times per day, 6 times per day, as needed, or any combination thereof for any therapeutically effective number of days. In some embodiments, the doses can be administered 1 hour apart, 2 hours apart, 3 hours apart, 4 hours apart, 6 hours apart, 8 hours apart, 12 hours apart, 24 hours apart, or any combination thereof. In some embodiments, the doses can be administered for one day, two days, 3 days, 4 days, 5 days, 6 days, 7 days, 10 days, 14 days, 3 weeks, 30 days, 2 months, 3 months, 4 months, 5 months, 6 months, 1 year, or any extended duration beyond one year, or any combination thereof. For example, the compositions can be administered as needed for any period of time, indefinitely, for the life of the subject treated.

In some embodiments, the composition can be administered in conjunction with at least one other therapeutic agent for the condition being treated. The amounts of the agents can be reduced, even substantially, such that the amount of the agent or agents desired is reduced to the extent that a significant response is observed from the subject. A significant response can include, but is not limited to, a reduction in fatigue, a reduction in an autoimmune response, an increase in weight loss, a reduction or elimination of nausea, a visible increase in tolerance, a faster response to the treatment, a more selective response to the treatment, or a combination thereof. In some embodiments, the methods taught herein can further include the administration of an antibiotic, an anti-emetic, an anticholinergic, an antispasmodic, or an anticancer agent.

Antibiotics can include, for example, aminoglycosides, ansamycins, carbacephem, carbapenems, cephalosporins (first through fifth generation), glycopeptides, lincosamides, macrolides, monobactams, penicillins, penicillin combinations, polypeptides, quinolones, sulfonamides, tetracyclines, and drugs against mycobacteria. In some embodiments, the antibiotic is selected from the group consisting of natural penicillin, cephalosporin, amoxicillin, ampicillin, clavamox, polymyxin, tetracycline, chlortetracycline, doxycycline, chloramphenicol, erythromycin, oleandomycin, streptomycin, gentamicin, kanamycin, tombramycin, nalidixic acid, rifamycin, rifampicin, prontisil, gantrisin, trimethoprim, isoniazid, para-aminosalicylic acid, and ethambutol. One of skill will appreciate that subgroups of this group can be desired in some embodiments. Anti-emetics can include, for example, anticholinergic agents, antidopaminergic agents, 5-HT3 antagonists, H1 antihistamines, cannabinoids, corticosteroids, and benzodiazepines. In some embodiments, the anti-emetics can be selected from the group consisting of benzodiazepines such as diazepam or lorazepam; 5-HT3 receptor antagonists such as ondansetron, tropisetron, granisetron, and dolasetron. Antispasmodics can include, for example, anticholinergics such as dicyclomine and hyoscyamine, as well as mebeverine and papaverine, for example. Anticancer agents can include, for example, alkylating agents, antimetabolites, anthracyclines, plant alkaloids, topoisomerase inhibitors, and other antitumor agents. One of skill will appreciate that the agents listed above can be used alone, or in combination, in some embodiments. For example, chemotherapy and anti-emetics can be administered together. And, anti-emetics can be administered together, such as a combination of corticosteroids and a second anti-emetic such as an antihistamine, anticholinergic, benzodiazepine, cannabinoid, or an anti-dopaminergic agent.

Combinations therapies can be administered, for example, for 30 minutes, 1 hour, 2 hours, 4 hours, 8 hours, 12 hours, 18 hours, 1 day, 2 days, 3 days, 4 days, 5 days, 6 days, 7 days, 8 days, 9 days, 10 days, 2 weeks, 3 weeks, 4 weeks, 6 weeks, 3 months, 6 months 1 year, any combination thereof, or any amount of time considered necessary by one of skill. In some embodiments, the combination therapies can be administered by the subject being treated on an as-needed basis. The agents can be administered concomitantly, sequentially, or cyclically to a subject. Cycling therapy involves the administering a first agent for a predetermined period of time, administering a second agent or therapy for a second predetermined period of time, and repeating this cycling for any desired purpose such as, for example, to enhance the efficacy of the treatment. The agents can also be administered concurrently. The term “concurrently” is not limited to the administration of agents at exactly the same time, but rather means that the agents can be administered in a sequence and time interval such that the agents can work together to provide additional benefit. Each agent can be administered separately or together in any appropriate form using any appropriate means of administering the agent or agents.

The compositions taught herein can be used in co-administrations with nutritional therapy or rehydration therapies. In some embodiments, the composition can be co-administered with at least one other nutritional and/or rehydrating agent for aiding recovery from a health imbalance, or to maintain a health balance. Examples of rehydrating agents can include, but are not limited to, GATORADE and other electrolyte drinks, oral rehydration solutions (ORSs) generally, new oral rehydration solution (N-ORS), SEURO ORAL, PEDIAONE, and PEDIALYTE. Examples of nutritional supplements can include, but are not limited to, zinc sulfate, salted rice water, salted yogurt-based drinks, and vegetable or chicken soup with salt. Such health imbalances can include, but is not limited to, dehydration, malnutrition, electrolyte imbalance, vitamin deficiency, food hypersensitivities, stress induced diarrhea, abdominal cramping, and alcohol hangover, or a combination thereof. In some embodiments, the methods taught herein can further include the administration of oral rehydrating or nutritional agents such as sodium, potassium, dextrose, fructose, glucose, magnesium, zinc, selenium, vitamin A, Vitamin D, Vitamin C, dietary fiber, and combinations thereof. The amounts and ratios of the agents to the composition can be substantially varied to provide prophylaxis, therapy or maintenance of healthful balance. Ratios of the compositions herein to the nutritional agents or rehydration agents can range, for example, from about 1:100 to about 100:1, from about 1:50 to about 50:1, from about 1:40 to about 40:1, from about 1:30 to about 30:1, from about 1:20 to about 20:1, from about 1:10 to about 10:1, from about 1:5 to about 5:1, from about 1:4 to about 4:1, from about 1:3 to about 3:1, from about 1:2 to about 2:1, from about 1:1.5 to about 1.5:1, about 1:1, or any range therein. The ratios can be based on volume:volume, mass:volume, volume:mass, mass:mass, or molar:molar. It should be appreciated that the concentrations of the compositions taught herein can be the same or different than the concentrations of the nutritional agents or rehydration agents. And, it should also be appreciated that the concentrations and ratios of concentrations can be subjective to a particular administration, such that they can be independently selected according to the condition treated, objective sought, desired effect, and/or personal preference. The combinations can be administered under any regime taught herein for the administration of an agent or combination of agents.

One of skill will appreciate that several dosage forms may be used to deliver the compositions taught herein. For example, dosage forms can include a paste, powder, solution, emulsion, cream, or gel having a sufficient thickness to maintain prolonged tissue contact. Alternatively, the agents can be formulated as a suppository, a sponge, a tablet, a capsule, pessary, or an absorbent material impregnated with a solution, lotion, or suspension containing a binding system taught herein. Any such form of drug delivery system which will effectively deliver the agent to a tissue is intended to be included in the teachings herein.

In some embodiments, the compositions are encapsulated as a dosage form for controlling release of the agents, prolonging shelf-life of the agents, improving ease of administration orally, rectally, or vaginally, and the like, as well as a timed-release or pulsed-delivery.

One of skill will appreciate that there are several known methods of encapsulation, each of which may be preferred in some embodiments. A capsule can be formed, for example, of a material selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polymerisates of acrylic or methacrylic esters, polyvinylacetate-phtalate and mixtures thereof. In some embodiments, the capsule can be soft and elastic, formed of a material selected from the group consisting of glycerin and sorbitol.

In some embodiments, the capsule can have the function of controlling a timed-release of the agent. The selection of the material, the thickness of the material, and the like, can be used to control timed-release of the agent.

In some embodiments, the capsule can have a plurality of compartments for a staged, time-release, or pulse-delivery, of one or more agents. Each of the compartments can have an independently selected material and or thickness to facilitate designing a desired timed-release of the one or more agents. Such designs can provide a release and delivery of the agent in intermittent intervals. A pulsed delivery, for example, may be provided by formulating the agent into individual layers, or compartments, interspaced with inactive layers of dissolvable coatings, or by using different encapsulation materials.

In some embodiments, the one or more agents can be released at once, or in stages, concurrently or sequentially, in minutes or hours. In some embodiments, the release occurs in about 5 minutes, about 10 minutes, about 15 minutes, about 20 minutes, about 30 minutes, about 1 hour, about 2 hours, about 3 hours, about 4 hours, about 6 hours, about 8 hours, about 12 hours, about 24 hours, or any range therein in increments of an hour. In some embodiments, the release occurs within about 1 hour to about 4 hours. In some embodiments, a first release occurs within about 1 hour to about 4 hours, and a second release within about 2 hours to about 8 hours.

### Articles of Manufacture

Articles of manufacture that encompass finished, packaged and labelled products are provided. The articles of manufacture include the appropriate unit dosage form in an appropriate vessel or container such as, for example, a glass vial or other container that is hermetically sealed. In the case of dosage forms suitable for oral administration, the active ingredient, e.g. one or more agents including a dosage form taught herein, may be suitable for administration orally, rectally, or the like.

As with any such product, the packaging material and container are designed to protect the stability of the product during storage and shipment. In addition, the articles of manufacture can include instructions for use or other information material that can advise the user such as, for example, a physician, technician or patient, regarding how to properly administer the composition as a prophylactic, therapeutic, or ameliorative treatment of the disease of concern. In some embodiments, instructions can indicate or suggest a dosing regimen that includes, but is not limited to, actual doses and monitoring procedures.

In some embodiments, the articles of manufacture can comprise one or more packaging materials such as, for example, a box, bottle, tube, vial, container, sprayer, insufflator, intravenous (I.V.) bag, envelope, and the like; and at least one unit dosage form of an agent comprising an extract taught herein within the packaging material. There can be a first composition comprising at least one unit dosage form of an agent comprising a binding system as taught herein within the packaging material, and optionally, a second composition comprising a second agent such as, for example, any other bioactive agent that may be administered in combination with the binding system, or any prodrugs, codrugs, metabolites, analogs, homologues, congeners, derivatives, salts, solvates, and combinations thereof. In some embodiments, the articles of manufacture may also include instructions for using the composition as a diagnostic, prophylactic, therapeutic, or ameliorative treatment for the condition of concern. In some embodiments, the instructions can include informational material indicating how to administer the systems for a particular use or range of uses, as well as how to monitor the subject for positive and/or negative responses to the systems.

In some embodiments, the article of manufacture can include a substantially anhydrous binding system. For example, a kit can be assembled which includes the anhydrous binding system comprising an anhydrous tannin with instructions combining the tannin with and an anhydrous reactive species generating component that forms a therapeutically, prophylactically, or nutritionally useful composition upon hydration.

Kits for the maintaining or restoring of digestive homeostasis are provided herein. In these embodiments, the kits can include the polyphenol component and/or the reactive oxygen species in a wet or dry form. Optionally, the kits can include instructions for use in treating a subject. The instructions can include, for example, instructions on diluting the composition to a desired concentration and administration according to suggested dilution factors on the basis of ages and weights of subjects, as well as known conditions and target sites. The suggested dilution factors can be selected from the ranges taught herein. In some embodiments, the kits comprise a dry, stable form of the composition components. For example, the kits can comprise a dry form of a polyphenol component, such as one polyphenol, a combination of polyphenols, or an extract of a plant tissue having polyphenols. Moreover, the kits can also comprise a dry form of a hydrogen peroxide generating material that functions to generate an effective amount of an exogeneous reactive oxygen species, wherein the reactive oxygen species includes a component selected from the group consisting of hydrogen peroxide, superoxide anion, singlet oxygen, and a hydroxyl radical. In these embodiments, the composition can be at least substantially free of active endogeneous oxidative enzymes and catalytic substances that cause degradation of the composition.

### Example 1. The Tannin-Hydrogen Peroxide Compositions are a Stable Binding System

This experiment combines hydrogen peroxide with gallic acid, tannic acid, a pomegranate husk extract, and a green tea extract to study the stability of the combinations. Since tannins are sometimes referred to as esters of gallic acid, gallic acid itself was studied as a basic building-block of the tannin compositions taught herein. Since gallic acid itself is effective and stable, as well as representative tannins, one of skill will appreciate that the tannins as a class are enabled by the teachings set-forth herein.

Measuring the Amount of Hydrogen Peroxide that Remains Bound to the Polyphenol

One of skill knows that hydrogen peroxide does not exist in a pure, solid form under normal conditions, for example, ambient conditions. However, this example shows that the hydrogen peroxide can exist in dry form when in association with the model compounds and plant extracts, and the compositions have been isolated in a dry form as proof. Art-recognized procedures, such as those set-forth at least in U.S. Pat. Nos. 3,860,694; 3,864,454; 4,171,280; and 4,966,762, were used as a guide for this study.

The model compounds were used to show that the compounds include hydrogen peroxide, the reactive oxygen species component, in a relatively stable association with the polyphenol component. As discussed, one of skill will appreciate that hydrogen peroxide in a free form, for example, would otherwise quickly degrade. The polyphenols were provided from model compounds or plant extracts. A dry form of the compositions was made between (i) gallic acid (a model polyphenol building block from Sigma-Aldrich) and hydrogen peroxide; (ii) tannic acid (a model polyphenol component from Sigma-Aldrich) and hydrogen peroxide; (iii) pomegranate husk extract and hydrogen peroxide; and, (iv) green tea extract and hydrogen peroxide, using the procedures taught herein, including:
i. adding a solution of 35% hydrogen peroxide slowly into each of the gallic acid powder, tannic powder, pomegranate husk extract powder, or green tea extract powder. The adding can be done in a glass dish or beaker at 45-65° C. under constant, gentle mixing;
ii. creating a dry form of the composition by continuing the heating under the constant, gentle mixing until fine dry granules or hard amorphous chunks form;
iii. crushing the granules or chunks into a powder, which is the dry form; dissolving the powder into water, knowing that the dry forms will not have stable, free hydrogen peroxide, such that the dissolved powder will carry only the stabilized hydrogen peroxide associated with the model compounds or extracts; and,
iv. measuring the total hydrogen peroxide concentration associated with the model compounds or extracts in the dry form.

The hydrogen peroxide concentration measurements were taken using standard methods to determine the amount of hydrogen peroxide that bound to the model compounds or extracts in the dry form. It was found that (i) about 3.0% hydrogen peroxide bound to the gallic acid (a model polyphenol building block) by total dry wt; (ii) about 2.5% hydrogen peroxide bound to the tannic acid (a model polyphenol component) by total dry wt; (iii) about 1.8% hydrogen peroxide bound to the pomegranate husk extract by total dry wt; and, (iv) about 2.0% hydrogen peroxide bound to the green tea extract by total dry wt. To measure the hydrogen peroxide levels, a standard, WATERWORKS peroxide test strip method was used having a test sensitivity of 0.5, 2, 5, 10, 25, 50, 100 ppm, available from Industrial Test Systems, Inc., Rock Hill, S.C. 29730.

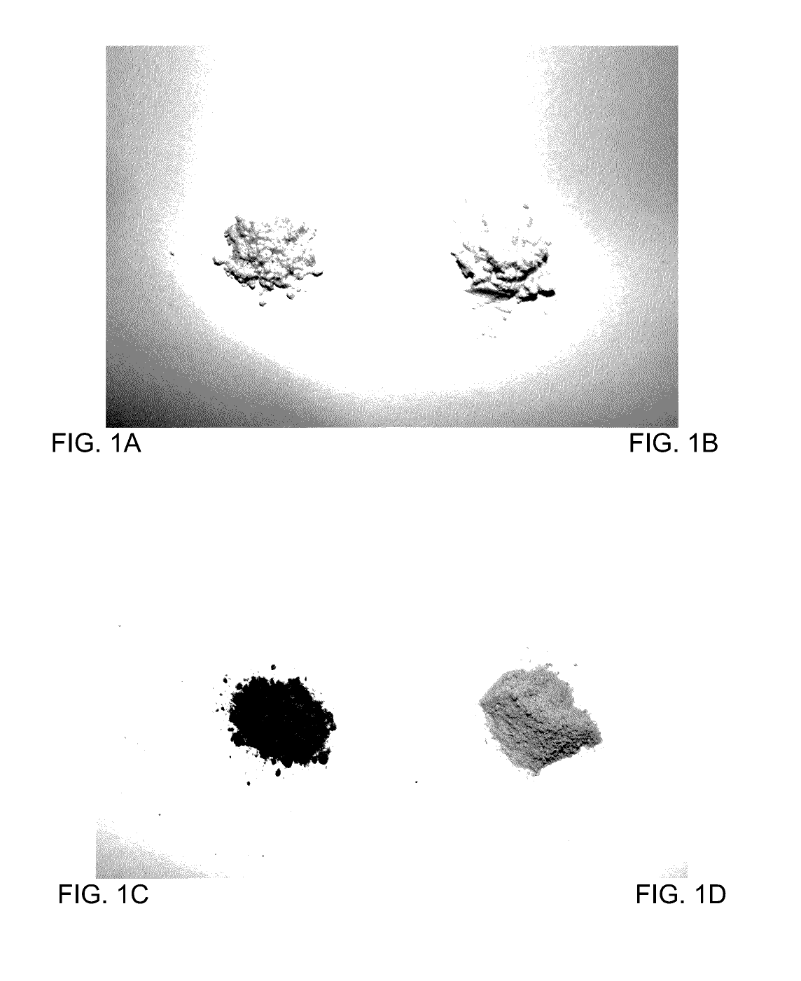

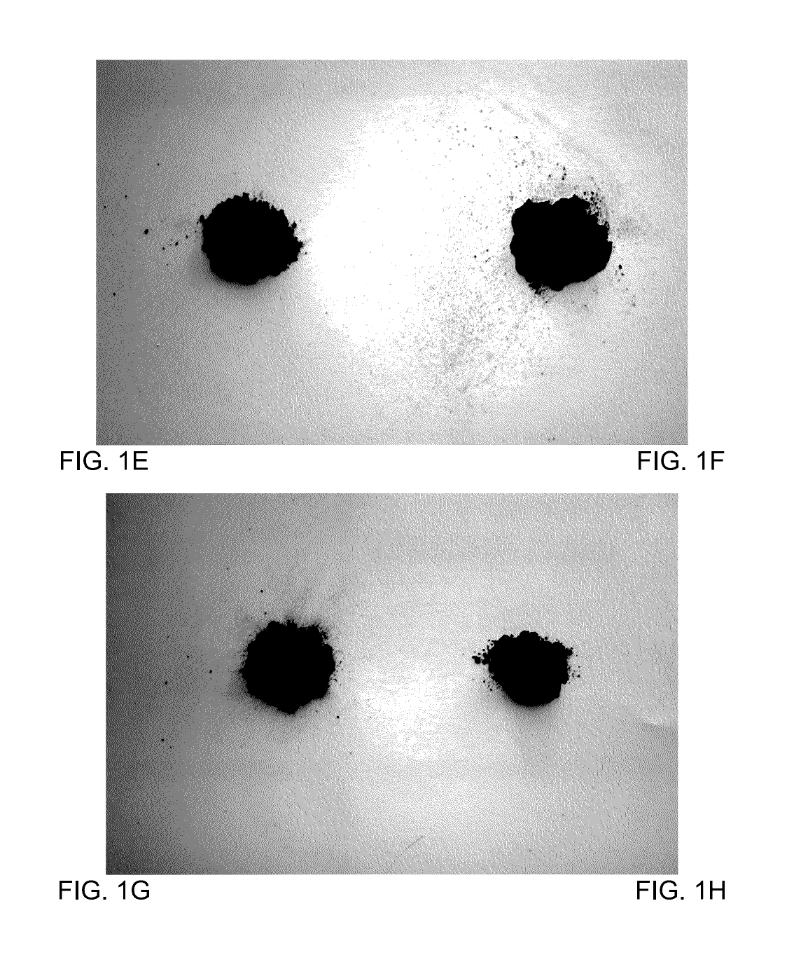

FIGS. 1A-1H are photographs of the dry forms of (A) gallic acid (a model polyphenol building block) bound to hydrogen peroxide; (B) gallic acid alone; (C) tannic acid (a model polyphenol) bound to hydrogen peroxide; (D) tannic acid alone; (E) pomegranate husk extract bound to hydrogen peroxide; (F) pomegranate husk extract alone; (G) green tea extract bound to hydrogen peroxide; and (H) green tea extract alone, according to some embodiments. As can be seen, the dry compositions exist and do contain a stable amount of hydrogen peroxide in an amount ranging from about 1.8% to about 3.0%, indicating the stabilizing association between the combined model compounds and extracts with the hydrogen peroxide. One of skill will appreciate that, surprisingly, the compositions contain a substantial amount of a stabilized hydrogen peroxide that is carried with the model compounds or extracts as a dry form.

The Stability of the Hydrogen Peroxide in the Combination is Greater in an Aqueous Solution than the Stability of the Hydrogen Peroxide Alone in the Aqueous Solution

This method tests the stability of the hydrogen peroxide in the combination. The testing methods used follow the standard procedures set-forth by the Clinical and Laboratory Standards Institute (CLSI) and US Pharmacopeia.
i. E. coli was chosen as the bacteria to challenge the stability of the bound compositions and the free hydrogen peroxide.
ii. The hydrogen peroxide concentration was matched to the selected bacteria in order to form a useful curve representing hydrogen peroxide degradation over time for the samples. As such, the hydrogen peroxide was varied from 62.5 ppm to 500 ppm on a fixed E. coli concentration of 10⁶ CFU/ml, and a concentration of 125 ppm was chosen as the initial hydrogen peroxide level used to challenge the E. coli over time.
iii. A ratio of 1:1 of the hydrogen peroxide to each of the model compounds and plant extracts was used to form each bound composition, such that 125 ppm of each plant extract was combined with the 125 ppm of the hydrogen peroxide.
iv. The free hydrogen peroxide was added at a concentration of 125 ppm as a control to show the relative stability of the hydrogen peroxide alone in the aqueous solution as compared to the bound compositions.

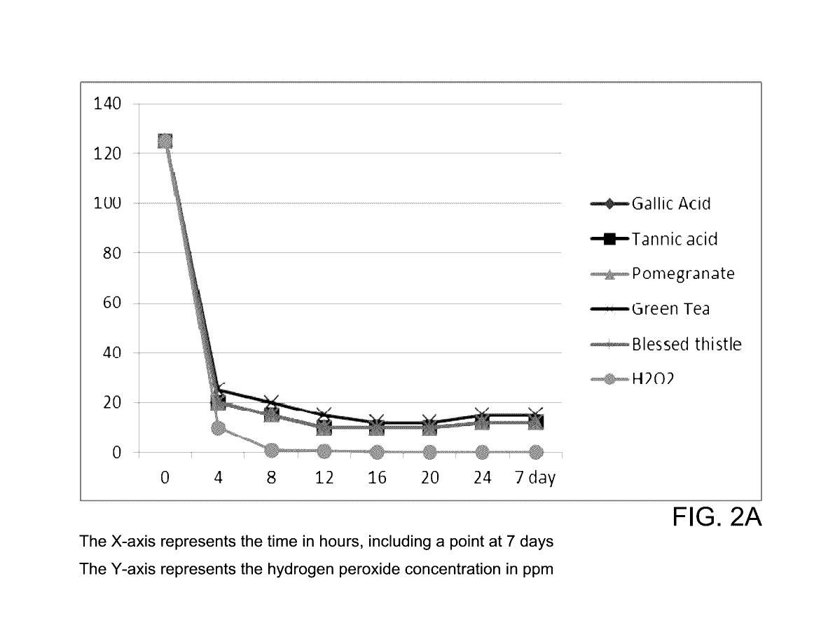

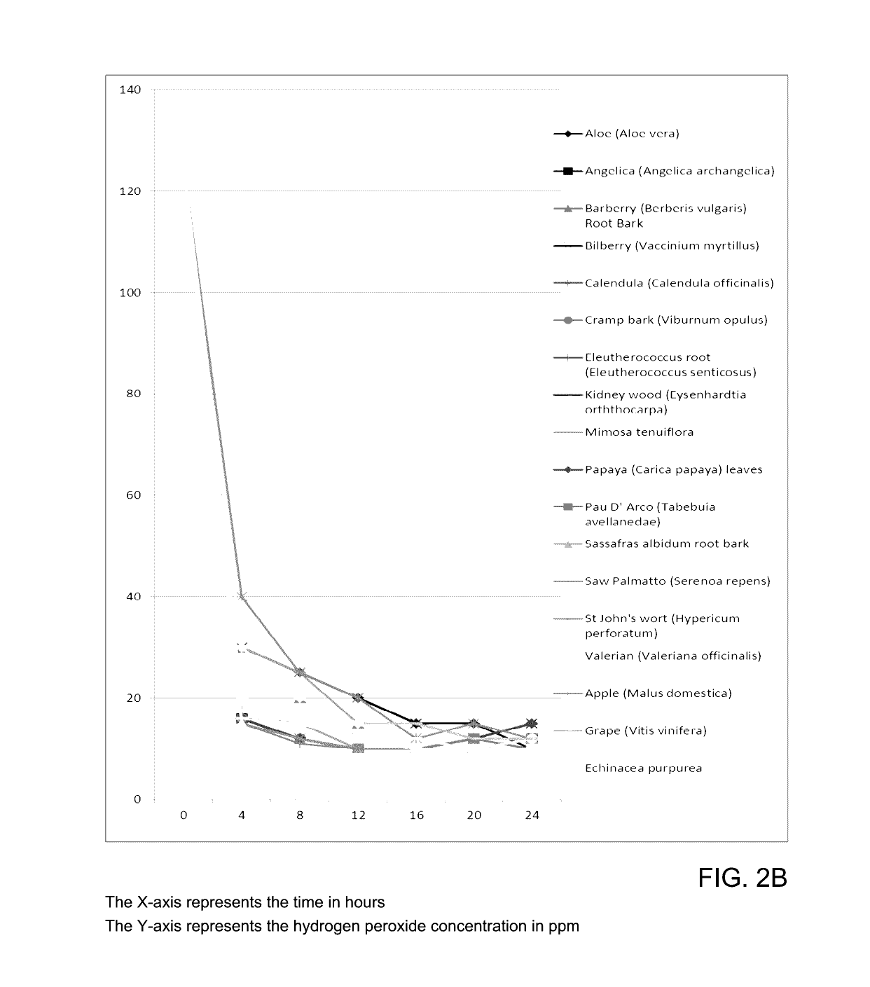

FIGS. 2A and 2B show that the stability of the hydrogen peroxide in the combination is consistently, substantially greater in an aqueous solution than the stability of the hydrogen peroxide alone in the aqueous solution, according to some embodiments. FIG. 2A compares stabilities of free hydrogen peroxide to hydrogen peroxide bound to each of: gallic acid (a model polyphenol building block), tannic acid (a model polyphenol), pomegranate husk extract, green tea extract, and Blessed thistle extract. FIG. 2B shows very similar and consistent stabilities when comparing free hydrogen peroxide to hydrogen peroxide bound to a wide variety of species of plants: Aloe, Angelica, Barberry Root Bark, Bilberry, Calendula, Cramp bark, Eleutherococcus root, Kidney wood, Mimosa tenuiflora, Papaya leaves, Pau D'Arco, Sassafras albidum root bark, Saw Palmatto, St. John's wort, Valerian, Apple, Grape, Echinacea purpurea, Grape seed extract, and Blueberry. In both FIGS. 2A and 2B, there are curves that cannot be identified well individually, as they are identical and overlapping. The free hydrogen peroxide curve does not overlap with any of the bound compositions beyond the 4 hour mark. Table 1 provides data used to produce the curves in the overlap for clarity.

TABLE 1

| | Hours | | | | | | |
|---|---|---|---|---|---|---|---|
| | 0 | 4 | 8 | 12 | 16 | 20 | 24 |
| Aloe (Aloe vera) | 125 | 30 | 25 | 20 | 15 | 15 | 10 |
| Angelica (Angelica archangelica) | 125 | 16 | 15 | 12 | 10 | 10 | 12 |
| Barberry (Berberis vulgaris) | 125 | 30 | 20 | 15 | 10 | 10 | 12 |
| Root Bark | | | | | | | |
| Bilberry (Vaccinium myrtillus) | 125 | 30 | 25 | 20 | 15 | 12 | 15 |
| Calendula (Calendula officinalis) | 125 | 16 | 15 | 12 | 10 | 10 | 10 |
| Cramp bark (Viburnum opulus) | 125 | 16 | 12 | 10 | 10 | 12 | 12 |
| Eleutherococcus root | 125 | 16 | 12 | 10 | 10 | 10 | 10 |
| (Eleutherococcus senticosus) | | | | | | | |
| Kidney wood (Eysenhardtia | 125 | 16 | 12 | 10 | 10 | 10 | 10 |
| orththocarpa) | | | | | | | |
| Mimosa tenuiflora | 125 | 30 | 25 | 15 | 10 | 12 | 15 |
| Papaya (Carica papaya) leaves | 125 | 16 | 12 | 10 | 10 | 12 | 15 |
| Pau D' Arco (Tabebuia avellanedae) | 125 | 20 | 15 | 10 | 10 | 12 | 10 |
| Sassafras albidum root bark | 125 | 20 | 15 | 10 | 10 | 10 | 12 |
| Saw Palmatto (Serenoa repens) | 125 | 15 | 12 | 10 | 10 | 10 | 12 |
| St John's wort (Hypericum | 125 | 40 | 25 | 20 | 12 | 15 | 12 |
| perforatum) | | | | | | | |
| Valerian (Valeriana officinalis) | 125 | 20 | 15 | 12 | 10 | 10 | 12 |
| Apple (Malus domestica) | 125 | 15 | 11 | 10 | 10 | 10 | 10 |
| Grape (Vitis vinifera) | 125 | 30 | 20 | 15 | 15 | 12 | 12 |
| Echinacea purpurea | 125 | 16 | 15 | 12 | 12 | 10 | 10 |
| Grape seed extract | 125 | 30 | 20 | 15 | 10 | 10 | 10 |
| Blueberry (Vaccinium corymbosum) | 125 | 20 | 15 | 12 | 10 | 10 | 10 |
| H2O2 | 125 | 15 | 0 | 0 | 0 | 0 | 0 |

The results were quite impressive and surprising, as the free hydrogen peroxide degraded quickly to near 0.0 ppm each time within about the first 8 hours, whereas each of the bound compositions maintained at least 10 ppm or greater for the total duration of the study, which was limited due to time constraints. As such, it was observed that the stabilities were maintained at a concentration of at least 10 ppm or greater for at least 24 hours, a concentration sufficient to maintain bactericidal activity in water. FIG. 2A shows that at least 7 days of stability remained present in at least the samples that were afforded the at least 7 days of testing. In fact, potencies have been observed to remain in the compositions when challenged for at least 30 days, and the original batches have shown to remain potent for at least 90 days, in some cases.

### Example 2. Activity Evidence to Support the Surprising, Synergistic Results

This experiment shows that the binding systems have an increased activity over either the tannin component or the hydrogen peroxide alone. The testing methods used follow the procedures set-forth by the Clinical and Laboratory Standards Institute (CLSI) and US Pharmacopeia.

E. coli was chosen as the bacteria to challenge the antibacterial activity of the bound compositions and the free hydrogen peroxide. A range of E. coli concentrations, ranging from 10-10⁶ CFU/ml were used for the study. A concentration of 100 ppm was chosen as the initial hydrogen peroxide level used to challenge the E. coli over time in both the free hydrogen peroxide and the bound compositions. A ratio of 1:1 of the hydrogen peroxide to the plant extract species was used in each bound composition, such that 100 ppm of each plant extract was combined with the 100 ppm of the hydrogen peroxide. The free hydrogen peroxide was added at a concentration of 100 ppm as a control to show the relative antibacterial activity of the hydrogen peroxide alone in the aqueous solution as compared to the bound compositions.

Table 2 compares the antibacterial activities of each of the model compounds and extracts alone, without the formation of the bound compositions: gallic acid (a model polyphenol building block), tannic acid (a model polyphenol), pomegranate husk extract, and green tea extract were each used to challenge the E. coli alone. Each were added into Muller-Hinton broth at a concentration of 100 ppm and allowed to challenge the E. coli for 24 hours at 37° C. As shown in the table, none of the model compounds or extracts showed any significant potency alone when challenging the E. coli. In the table, “+” indicates that there was positive growth of the E. coli despite the challenge of the particular model compound or extract.

TABLE 2

| | | | 1% polyphenol | 1% polyphenol |
|---|---|---|---|---|
| | 1% Tannic | 1% Gallic | Pomegranate | Green Tea |
| | Acid | Acid | extract | Extract |
| | Muller - | Muller - | Muller - | Muller - |
| E coli | Hinton | Hinton | Hinton | Hinton |
| CFU/ml | Broth | Broth | Broth | Broth |
| 10 | + | + | + | + |
| 10² | + | + | + | + |
| 10³ | + | + | + | + |
| 10⁴ | + | + | + | + |
| 10⁵ | + | + | + | + |
| 10⁶ | + | + | + | + |

“+” means positive identification of bacterial growth

Table 3 compares the antibacterial activities of free hydrogen peroxide to hydrogen peroxide bound to each of: gallic acid (a model polyphenol building block), tannic acid (a model polyphenol), pomegranate husk extract, and green tea extract. Each were added into Muller-Hinton broth at a concentration of 100 ppm and allowed to challenge the E. coli for 24 hours at 37° C. As shown in the table, all of the E. coli concentrations were killed by each of the bound compositions, yet all of the E. coli concentrations managed to survive under exposure to the free hydrogen peroxide alone.

TABLE 3

| | Pomegranate | Green Tea | Tannic | Gallic | |
|---|---|---|---|---|---|
| | Extract + | Extract + | Acid + | Acid + | 100 |
| E coli | 100 ppm | 100 ppm | 100 ppm | 100 ppm | ppm |
| CFU/ml | H₂O₂ | H₂O₂ | H2O2 | H₂O₂ | H₂O₂ |
| 10⁵ | − | − | − | − | + |
| 10⁴ | − | − | − | − | + |
| 10³ | − | − | − | − | + |
| 10² | − | − | − | − | + |

“+” means identifiable bacterial growth.
“−” means no bacterial growth

The results were quite impressive and surprising, as they show that the bound composition has an increased activity over either the polyphenolic component or the hydrogen peroxide alone by at least 4 orders of magnitude, using the test of relative activity as the bactericidal effect on E. coli. The model compounds and plant extracts did not contribute a cumulative effect but, rather, a surprising and unexpected synergistic effect.

### Example 3. Selective Binding of the Binding Systems in a Lipopolysaccharide (LPS) Model

This experiment is designed to show that a composition having a combination of tannins and hydrogen peroxide selectively binds to, and reduces, the infectivity or propogation of virus, bacteria, yeast or fungi.

Upon enzymatic bioactivation by pathogens or damaged tissues, the compositions exhibit increased binding inactivation of endotoxins, such as lipopolysaccharides, and exotoxins, such as cholera toxin, botulism, and other virulence factors of bacteria that are pathogenic to a subject, human or non-human. The selectivity is likely due to the polyphenol-hydrogen peroxide aggregates being generally unreactive with digestive enzymes such as proteases and peptidases that split proteins into their monomers, the amino acids, lipases that split fat into three fatty acids and a glycerol molecule, carbohydrases that split carbohydrates such as starch and sugars into simple sugars, or nucleases that split nucleic acids into nucleotides. As such, the compositions are binding systems that selectively activate respond to target specific enzymes and exhibit orders of magnitude (500× or more) differential between active and passive states providing focused toxin binding, pathogen or damage specific effects with a reduction in undesirable collateral effects. In the animal body, the activated binding systems can actively form glycosydic bonds, as well as complex proteins and amino acids. The binding of the phenolic compound to, for example, glucuronic acid or other glucose moieties can neutralize the activity of lipopolysaccharides and other important toxins.

### Experimental

First, a serial dilution of a binding system of tannins and hydrogen peroxide was used to show binding selectivity. The tannins used were rich in gallotannins and were carried in an extract of Chinese Gall. The tannin-hydrogen peroxide combination (“the binding system”; from 0 to 10 μg/ml) was incubated with a lipopolysaccharide (LPS), then reacted with standard polymixin B with and without horseradish peroxidase at 37° C. It was observed that, when combined with horseradish peroxidase, the binding system exhibited over 500× increase in lipopolysaccharide binding compared to the binding system without horseradish peroxidase as determined by ELISA measurements of polymixin B binding inhibition test.

Next, we performed an anti-cholera toxin B antibody binding inhibition experiment. A serial dilution of the binding system was combined with cholera toxin, then reacted with anti-cholera toxin B antibody with and without horseradish peroxidase at 37° C. The result showed that the combination of horseradish peroxidase and the binding system exhibited over 500× increase over the binding system without the peroxidase in anti-cholera toxin B antibody binding as determined by ELISA measurements.

These results clearly demonstrate a surprising and extraordinarily efficient binding of two distinctly different toxins upon enzyme activation. The large differential in activity indicates the viability of delivering a tannin-hydrogen peroxide binding system for a localized and aggressive remote activation by tissues, tissue conditions, or pathogens that express peroxidase enzymes or other site specific enzymes utilizing hydrogen peroxide or its decomposition products as a reaction promoting substrate.

### Example 4. The Binding Systems Effectively Inhibit the Growth of Four (4) Antibiotic-Resistant Bacteria: Clostridium difficile (ATCC 43598), Enterococcus faecalis (VRE) (ATCC 51299), Staphylococcus aureus (MRSA) (ATCC 22592), and Klebsiella pneumoniae (CRE) (ATCC BAA2146)

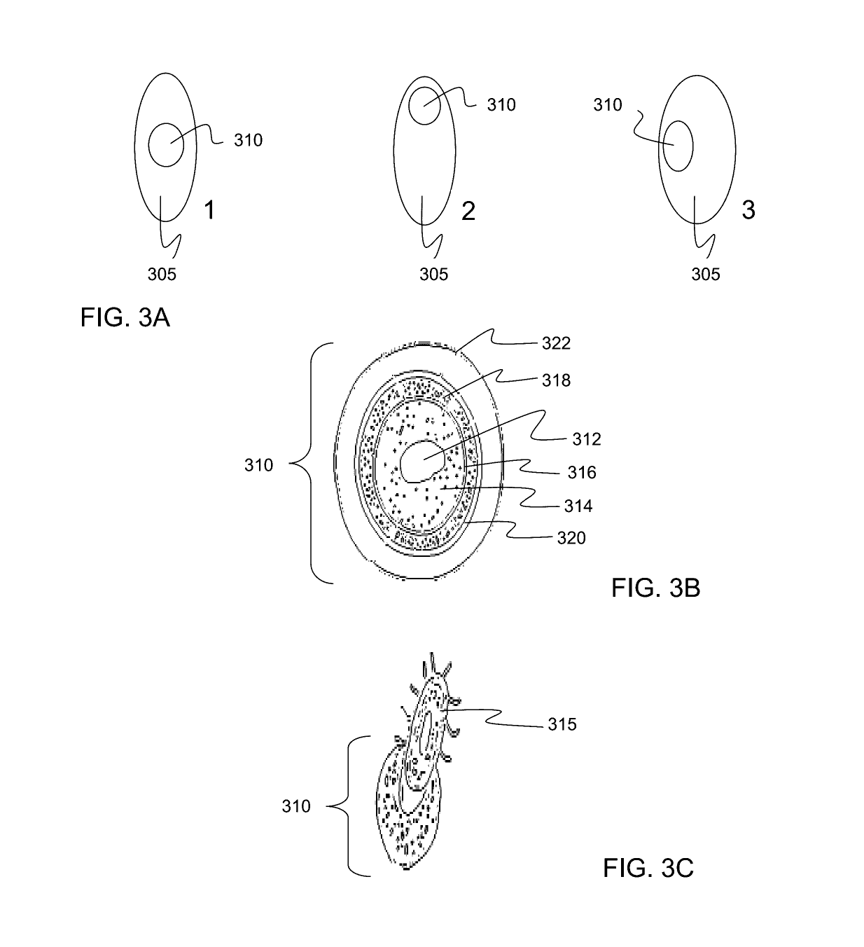

FIGS. 3A-3C illustrate an endospore and germination, according to some embodiments. Antibiotic-resistant bacterial can include endospores. An endopore 310 has a structure within a parent cell 305 that protects the bacteria from conditions in which it may not otherwise survive. The endospore 310 has a structure, as shown in FIG. 3A, based on 3 main morphologies: central 3A1; terminal 3A2, and lateral 3A3. As shown in the cross-section of the endospore in FIG. 3B, in the formation of the endospore, a portion of the cytoplasm 314 and a copy of the bacterial chromosome in the nucleus 312 undergoes dehydration, and is surrounded by a three-layered covering: the core wall 316, the spore coat 320, and the exosporium 322, having a cortex 318 between the core wall 316 and the spore coat 320. The remaining part of cytoplasm 314 and cell wall degenerate. The resulting endospore 310 can then tolerate extreme environmental conditions and remain viable for a very long time, for example, many years, after which the endospore 310 can absorb water, swell and release a new bacterium 315 from the endospore 310 as shown in FIG. 3C. The bacteria 315 has a new cell wall and functions as a typical bacterial cell. In some embodiments, the methods and compositions provided herein can at least inhibit the onset, inhibit the release of a bacterium from, and/or kill a central endospore. In some embodiments, the methods and compositions provided herein can at least inhibit the onset, inhibit the release of a bacterium from, and/or kill a terminal endospore. In some embodiments, the methods and compositions provided herein can at least inhibit the onset, inhibit the release of a bacterium from, and/or kill a lateral endospore.

The binding systems effectively inhibit the growth of antibiotic-resistant bacteria. The minimum inhibitory concentration (MIC) of a binding system taught herein was determined using (4) antibiotic-resistant bacteria: Clostridium difficile, Enterococcus faecalis (VRE; vancomycin-resistant enterocci), Staphylococcus aureus (MRSA; methicillin-resistant S. aureus), and Klebsiella pneumoniae (CRE; carbapenem-resistant Enterobacteriaceae).

### The Test Solution

The test solution (“the binding system”) contained a ratio of green tea leaf extract (GT) to pomegranate extract (POM) that was approximately 1:3 GT:POM. The ratio contained approximately 1100 micrograms total dry weight of dessicated pomegranate and green tea extract dissolved in a solution of 0.05% hydrogen peroxide in 15 ml purified water. Unused and undiluted solutions of the composition from the same lot were tested for hydrogen peroxide concentration using standard methodologies, described herein, verifying an unchanged ratio of peroxide to polyphenols. The free hydrogen peroxide at the fully diluted oral concentration was well below its conventionally accepted minimum inhibitory concentration for most bacteria.

The composition was tested for stability. Consistent with the methods taught herein, the composition was dessicated to a gummy solid with slow heating in a glass dish or beaker at 45-65° C. under constant, gentle mixing, along with vacuum dessication to degrade free hydrogen peroxide. The composition was then rehydrated to its original liquid volume to determine the amount of hydrogen peroxide that was stable enough to remain in the composition. The composition retained a substantial concentration of a stable, hydrogen peroxide through the dessication and rehydration cycle, providing evidence that the binding system is stable.

A 1430 ug/ml (dry weight active) of the binding system was diluted 1:1 in reverse-osmosis water until ten dilutions were produced for use in this experiment: 50%, 25%, 12.5%, 6.25%, 3.125%, 1.563%, 0.781%, 0.391%, 0.195%, and 0.098%.

### The Bacteria

Each of the four bacteria were tested in the following manner, using the Clostridium difficile as an example: After being cultured overnight, C. diff. ribotype 017 (ATCC 43598), for example, was diluted to a target concentration of approximately 1×107 CFU/ml, and a 150 uL volume of the bacterium was added to an 8 ml sterile test tube containing thioglycallate broth. Using three replicates (runs), these dilutions were added to the test tubes, which were incubated in a controlled oven for 48 hours at 36° C. (+/−1° C.). At the end of 48 hours of incubation, the test tubes were removed from the oven and evaluated for growth of the bacteria; visible turbidity in the test tube denotes growth, while no turbidity denotes inhibition of the bacterium.

Tables 4 and 5 show that growth of C. diff, for example, was inhibited at dilutions of 50% (720 ug/ml), 25% (360 ug/ml), 12.5% (180 ug/ml), and 6.25% (90 ug/ml) of the binding system. C. diff had the highest MIC of the four antibiotic-resistant organisms tested and it's growth was inhibited at concentrations well below the concentrations of the binding system used in human studies.

TABLE 4

| | Average | | | Positive | Negative |
|---|---|---|---|---|---|
| Microorganism | CFU/well | Run | MIC, % | Control | Control |
| S. aureus | 1.43E+06 | 1 | 0.391 | + | − |
| ATCC 22592 | | 2 | 0.391 | + | − |
| (MRSA) | | 3 | 0.781 | + | − |
| E. faecalis | 1.48E+06 | 1 | 3.125 | + | − |
| ATCC 51299 | | 2 | 1.563 | + | − |
| (VRE) | | 3 | 1.563 | + | − |
| C. difficile | 2.20E+06 | 1 | 6.250 | + | − |
| ATCC 43598 | | 2 | 6.250 | + | − |
| | | 3 | 6.250 | + | − |
| K. pneumoniae | 1.37E+06 | 1 | 0.195 | + | − |
| ATCC BAA2146 | | 2 | 0.195 | + | − |
| (CRE) | | 3 | 0.195 | + | − |

TABLE 5

| | | Percent test substance | | | | | | | | | |
|---|---|---|---|---|---|---|---|---|---|---|---|
| | | (ug/ml) | | | | | | | | | |
| | | 50.0 | 25.0 | 12.5 | 6.250 | 3.125 | 1.563 | 0.781 | 0.391 | 0.195 | 0.098 |
| Microorganism | Run | (720) | (360) | (180) | (90) | (45) | (22.4) | (11.2) | (5.6) | (2.8) | (1.4) |
| S. aureus | 1 | − | − | − | − | − | − | − | − | + | + |
| ATCC 22592 | 2 | − | − | − | − | − | − | − | − | + | + |
| (MRSA) | 3 | − | − | − | − | − | − | − | + | + | + |
| E. faecalis | 1 | − | − | − | − | − | + | + | + | + | + |
| ATCC 51299 | 2 | − | − | − | − | − | − | + | + | + | + |
| (VRE) | 3 | − | − | − | − | − | − | + | + | + | + |
| C. difficile | 1 | − | − | − | − | + | + | + | + | + | + |
| ATCC 43598 | 2 | − | − | − | − | + | + | + | + | + | + |
| | 3 | − | − | − | − | + | + | + | + | + | + |
| K. pneumoniae | 1 | − | − | − | − | − | − | − | − | − | + |
| ATCC BAA2146 | 2 | − | − | − | − | − | − | − | − | − | + |
| (CRE) | 3 | − | − | − | − | − | − | − | − | − | + |

‘+’ indicates observable turbidity, microbial growth
‘−’ indicates no observable turbidity, no microbial growth

The binding system effect on S. aureus (not methicillin-resistant) and MRSA was tested in a separate study and showed an equivalent MIC at 0.391% (11.25 ug/ml), indicating non-involvement of resistance mechanism through the equal effect on the resistant and non-resistant forms. In fact, the MIC of the binding system with S. aureus provided an inhibition that was similar to RIFAXIMIN, a rifamycin antibiotic. The MICs of the binding system for each of the four antibiotic-resistant bacteria provides one of skill with the enablement needed to effectively control a wide range of hospital acquired infections (HAIs), for example, at even lower concentrations than that required to control the growth of C. diff.

As such, in view of the results, one of skill will appreciate that these findings show a bacteriostatic and bactericidal effect of the binding systems on a wide range of antibiotic-resistant bacteria.

### Example 5. The Binding Systems Effectively Treat Patients Having a C. diff. Infection

Two (2) binding systems were given to 7 patients in an open-label study that was monitored by 3 physicians to show the effectiveness of the systems on patients having C. diff. infections.

The test solution of Example 4 was used in this study.

### The Study

A total of 7 patients were presented with diarrhea and other gastrointestinal (GI) symptoms at a community hospital. These patients ranged from 1 month to 13 years of age. All patients had a positive culture for the C. diff toxin, although they also were diagnosed with additional GI conditions, such as Crohn's disease and ulcerative colitis (UC).

The two binding systems were administered at concentrations of 132 μg/ml and in doses ranging from 7 ml (925 ug dry wt of the binding system) to 14 ml (1850 ug dry wt of the binding system), the dose adjusted for the weight of the patient. The dosages were administered each day, once per day, for a period of time ranging from 14 days to 21 days, and symptoms were recorded before and after the administration period. Follow-up stool cultures for the presence of C. diff toxins were performed. 5 of the 7 patients completed the follow-up monitoring, and the results are presented in Table 6.

TABLE 6

| | | | | Stool | Stool | |
|---|---|---|---|---|---|---|
| | | | | culture for | culture for | |
| | | | | C. diff. | C. diff. toxin | Symptoms |
| | Age/Sex | | | toxin after | after | after |
| Patient | (Body Wt) | Symptoms | Diagnosis | administration | administration | administration |
| 1 | 7 yrs | Abdominal | C. diff., | positive | negative | all resolved |
| | male | pain, | enterocolitis | | | within 2 days |
| | (31 kg) | diarrhea>6 mos | | | | |
| 2 | 13 yrs | abdominal | C. diff., | positive | negative | abdominal |
| | male | pain, diarrhea, | Crohn's | | | pain, |
| | (39 kg) | vomiting, | disease | | | diarrhea, |
| | | fatigue, weight | | | | vomiting, |
| | | loss, growth | | | | fatigue, |
| | | failure | | | | weight loss, |
| | | | | | | growth failure |
| 3 | 5 mos | Diarrhea, | C. diff., | positive | positive | blood in stool |
| | female | blood in stool, | cow's milk | | | |
| | (6 kg) | cow's milk | intolerance, | | | |
| | | intolerance | enterocolitis | | | |
| 4 | 12 yrs | Diarrhea, | C. diff., | positive | negative | diarrhea, |
| | female | abdominal | enterocolitis, | | | rectal |
| | (39 kg) | pain, rectal | ulcerative | | | bleeding |
| | | bleeding, | colitis | | | |
| | | fatigue, weight | | | | |
| | | loss | | | | |
| 5 | 5 mos | Diarrhea, | C. diff., | positive | negative | all resolved |
| | male | vomiting, rectal | cow's milk | | | |
| | (7 kg) | bleeding | intolerance, | | | |
| | | | enterocolitis | | | |

Patient 1 was given 14 ml/day for 14 days (59.7 ug/kg/day);
Patient 2 was given 14 ml/day for 21 days (47.4 ug/kg/day);
Patient 3 was given 7.5 ml/day for 21 days (154.2 ug/kg/day);
Patient 4 was given 14 ml/day for 14 days (47.4 ug/kg/day); and,
Patient 5 was given 7.5 ml/day for 14 days (132.1 ug/kg/day).

4 Out of the 5 Patients that Completed Reported were Treated Successfully for the C. diff. Toxin.

As shown in Table 6, all 5 patients had a positive stool culture for the C. diff. toxin prior to consumption of the binding systems. At the end of the monitoring period, 4 out of the 5 had a negative stool culture for the C. diff. toxin. Moreover, diarrhea, abdominal pain, vomiting, and rectal bleeding were resolved completely in 2 out of the 5 patients. GI symptoms remained in 3 of the patients; however, these patients had concurrent Crohn's disease, intolerance to cow's milk protein, or ulcerative colitis, which can account for the symptoms that each of the patients noted. As such, one of skill will appreciate that these findings show a bacteriostatic and bactericidal effect of the binding systems on C. diff in the patients, as the MIC study shows a clear point at which exposure to the binding systems inhibits growth of the C. diff. (bacteriostatic), and in 4 of 5 patients the C. diff toxins were no longer present at all (bactericidal) at the end of the treatment period.

### Preclinical Studies

In a preclinical study, the binding system was a 1:1 ratio of POM:GT. A concentration used in humans can be 132 μg/ml, and this concentration was increased by a factor of 500/185 for piglets to be administered at 357 μg/ml. It was orally dispensed at 2 cc to newborn piglets having an E. coli infection and the results were determined after an 8 hour period. The E. coli infection was removed from the piglets and, moreover, it was observed that the ileum crypts were deeper in the treated piglets, suggesting that the binding system was not only effective at treating the infection, but it was also had a reparative and/or protective activity.

The experiments shown herein are for illustration and example only. One of skill can vary the experimental conditions and components to suit a particular or alternate experimental design. The experimental conditions can be in vitro or in vivo, or designed for any subject, for example, human or non-human. For example, animal testing can be varied to suit a desired experimental method. As such, one of skill will appreciate that the concepts can extend well-beyond the examples shown, a literal reading of the claims, the inventions recited by the claims, and the terms recited in the claims.

We claim:

1. A method of treating a subject that is hosting an antibiotic-resistant bacteria, the method comprising:
administering an effective amount of a formulation to a subject that is hosting an antibiotic-resistant bacteria, the formulation having a water soluble tannin combined with hydrogen peroxide in a pharmaceutically acceptable excipient;
wherein,
the tannin has a molecular weight ranging from about 170 Daltons to about 4000 Daltons;
the tannin:peroxide weight ratio ranges from about 1:1000 to about 10:1; and,
the formulation at least inhibits the growth of the antibiotic-resistant bacteria in the subject when compared to a second subject in a control group also hosting the antibiotic-resistant bacteria in which the formulation was not administered.

2. The method of claim 1, wherein the antibiotic-resistant bacteria is Clostridium difficile.

3. The method of claim 1, wherein the antibiotic-resistant bacteria is Enterococcus faecalis.

4. The method of claim 1, wherein the antibiotic-resistant bacteria is Staphylococcus aureus.

5. The method of claim 1, wherein the antibiotic-resistant bacteria is Klebsiella pneumoniae.

6. The method of claim 1, wherein the tannin is a catechin.

7. The method of claim 1, wherein the tannin is gallic acid, epigallic acid, or a combination thereof.

8. The method of claim 1, wherein the tannin is an ellagitannin.

9. The method of claim 1, wherein the tannin is punicalagin.

10. The method of claim 1, wherein the tannin is tannic acid.

11. A method of treating a gastrointestinal inflammation in a subject that is hosting the antibiotic-resistant bacteria, comprising:
administering an effective amount of a formulation to a subject that is hosting the antibiotic-resistant bacteria, the formulation produced from a process including
combining a water soluble tannin with hydrogen peroxide at a tannin:peroxide weight ratio that ranges from about 1:1000 to about 10:1, the tannin having a molecular weight ranging from about 170 Daltons to about 4000 Daltons;
removing free hydrogen peroxide from the combination; and,
mixing the combination of the tannin and the hydrogen peroxide with a pharmaceutically acceptable excipient to create the formulation;
wherein,
the administering includes selecting a desired concentration of the formulation for the administering; and,
the formulation relieves a gastrointestinal inflammation in the subject that is hosting the antibiotic-resistant bacteria when compared to a second subject in a control group also hosting the antibiotic-resistant bacteria in which the formulation was not administered.

12. The method of claim 11, wherein the antibiotic-resistant bacteria is Clostridium difficile.

13. The method of claim 11, wherein the antibiotic-resistant bacteria is Enterococcus faecalis.

14. The method of claim 11, wherein the antibiotic-resistant bacteria is Staphylococcus aureus.

15. The method of claim 11, wherein the antibiotic-resistant bacteria is Klebsiella pneumoniae.

16. The method of claim 11, wherein the tannin is a catechin.

17. The method of claim 11, wherein the tannin is a gallotannin, gallic acid, epigallic acid, or a combination thereof.

18. The method of claim 11, wherein the tannin is an ellagitannin.

19. The method of claim 11, wherein the tannin is a punicalagin.

20. A method of treating diarrhea in a subject that is hosting an antibiotic-resistant bacteria, comprising:
administering an effective amount of a composition to a subject that is hosting an antibiotic-resistant bacteria, the composition produced from a process including
combining a water soluble, hydrolysable tannin with hydrogen peroxide at a tannin:peroxide weight ratio that ranges from about 1:1000 to about 10:1, the tannin having a molecular weight ranging from about 170 Daltons to about 4000 Daltons;
wherein,
the administering includes selecting a desired concentration of the formulation for the administering; and,
the formulation relieves diarrhea in the subject that is hosting the antibiotic-resistant bacteria when compared to a second subject in a control group also hosting the antibiotic-resistant bacteria in which the formulation was not administered.

21. The method of claim 20, wherein the antibiotic-resistant bacteria is Clostridium difficile.

22. The method of claim 20, wherein the antibiotic-resistant bacteria is Enterococcus faecalis.

23. The method of claim 20, wherein the antibiotic-resistant bacteria is Staphylococcus aureus.

24. The method of claim 20, wherein the antibiotic-resistant bacteria is Klebsiella pneumoniae.

25. The method of claim 20, wherein the tannin is a gallotannin, gallic acid, epigallic acid, or a combination thereof.

26. The method of claim 20, wherein the tannin is a catechin.

27. The method of claim 20, wherein the tannin is an ellagitannin.

28. The method of claim 20, wherein the tannin is a punicalagin.

29. The method of claim 20, wherein the tannin is tannic acid.

30. A method of inhibiting the growth of an antibiotic-resistant bacteria, the method comprising:
contacting an antibiotic-resistant bacteria with a composition having a water soluble tannin combined with hydrogen peroxide;
wherein,
the tannin has a molecular weight ranging from about 170 Daltons to about 4000 Daltons;
the tannin:peroxide weight ratio ranges from about 1:1000 to about 10:1; and,
the composition inhibits the growth of the antibiotic-resistant bacteria when compared to a negative control group.
